(19) United States
(12) Patent Application Publication
(10) Pub. No.: US 2026/0276621 A1
(43) Pub. Date: Sep. 17, 2026

(54) METHOD AND APPARATUS FOR DETERMINING THE SECTION THICKNESS OF HISTOLOGICAL SECTIONS AND DETERMINING THE CONCENTRATION OF BIOLOGICAL SUBSTANCES IN HISTOLOGICAL SPECIMENS

(71) Applicant: TECHNISCHE UNIVERSITÄT DRESDEN, Dresden (DE)

(72) Inventors: Matthias MEINHARDT, Pesterwitz (DE); Adrian EHRENHOFER, Dresden (DE)

(73) Assignee: TECHNISCHE UNIVERSITÄT DRESDEN, Dresden (DE)

(21) Appl. No.: 19/140,994

(22) PCT Filed: Dec. 20, 2023
(86) PCT No.: PCT/EP2023/087035

§ 371 Date: Jun. 18, 2025

(30) Foreign Application Priority Data
Dec. 22, 2022 (DE) 10 2022 134 504.1

(51) Int. Cl. G01N 33/483; G01B 11/06

(52) U.S. Cl. CPC G01N 33/4833; G01B 11/06

(57) ABSTRACT

The invention relates to a method for determining a section thickness of a specimen section in medical histology. In a further aspect, the invention relates to a method for determining the concentration of a target substance in a tissue contained in the specimen section, in particular for the diagnostic determination of the concentration in medical specimen sections. In a further aspect, the invention relates to an apparatus for measuring the section thickness of a specimen section and optionally for determining the concentration of a target substance in the specimen section.

The invention relates to the field of medicine, diagnostics, histology (and sections of cell blocks), staining and evaluation of tissue sections and the measurement of the concentration of target substances in tissue sections. In particular, the invention relates to the field of medical histology and the diagnostic measurement of the concentration of target substances in tissue sections.

The invention relates to a method for determining a section thickness of a specimen section, comprising (a) measuring values generated from scattering and/or absorption by the section, (b) providing modeling of the section thickness of the specimen section based on calibration values, and (c) determining the section thickness of the specimen section by using the measured values and the modeling according to (b). The invention preferably relates to a method for determining a section thickness of a specimen section, comprising (a) measuring values generated from the scattering and/or absorption of a light beam by the specimen section using one or more light sources of different wavelengths, and (b) providing modeling of the section thickness of the specimen section based on calibration values, generated by measuring the scattering and absorption of a light beam by one or more calibration specimens using one or more light sources of different wavelengths, and (c) determining the section thickness of the specimen section by using the measured values according to (a) and modeling according to (b).

In a further aspect, the invention relates to an apparatus for measuring the section thickness of a specimen section and optionally for determining the concentration of a target substance in the section, comprising (a) one or more light sources of different wavelengths, (b) at least one sensor for measuring the scattering and/or absorption of a light beam, (c) an apparatus for positioning the section during the measurement, and (d) a computing unit for modeling the section thickness of a specimen section or for using the modeling in determining the section thickness of a specimen section.

In a further aspect, the invention relates to a slide for use in the method or apparatus, comprising a calibration specimen containing a target substance in two or more concentrations which are spatially separated from each other, onto which the histological specimen section can additionally be applied.

### BACKGROUND AND PRIOR ART

Histopathology involves the examination of tissue sections such as biopsies using special staining techniques and is an important diagnostic tool in medicine. It is used, for example, to assess malignancy and for early diagnosis of tumors, as well as in the detection of metabolic, parasitic bacterial and inflammatory diseases and thus for decision-making in the choice of therapy.

Since its introduction around 150 years ago, the method of histopathology has involved obtaining and producing sections of human or animal tissue and then using dyes to make the tissue structures or substances contained therein visible for examination under the microscope. The extraction, production and staining of the sections is referred to as histology and the sections obtained as histological specimens.

Four steps that are common to all methods of classical histology and histopathology are (i) preparation including fixation and embedding or freezing, (ii) sectioning, (iii) staining and (iv) microscopic evaluation of the sample.
(i) Various types of histological specimens are known that require different preparation, such as paraffin-embedded specimens or specimens made by frozen sectioning. The selection of the histological specimen and the associated preparation steps depends on the properties of the tissue, the structures to be examined in the tissue, their stability during dissection and preparation as well as the clinical situation and urgency. As a rule, paraffin-embedded specimens are used, wherein the tissue to be examined is fixed in formalin and embedded in paraffin.
(ii) In the next step, the embedded or frozen tissue is usually cut manually on the microtome. The section thickness is mechanically adjusted to a thickness between 1 and 9 μm, usually 1 to 5 μm. The section curls strongly during cutting and is also influenced by the tissue it contains. The section is then stretched on a water surface and transferred from there to a slide for further processing and examination. The precision of these devices varies greatly and is also subject to strong fluctuations due to manual operation. The fluctuations in the actual section thickness of the cut histological specimen can be more than 200%. In addition, the section thickness set on the microtome often differs considerably from the actual section thickness of the specimen. This was investigated in a study by Masuda et al., 1998. In the study, a confocal laser scanning microscope was used to examine cross-sections of numerous paraffin-embedded specimen sections produced using routine methods with a section thickness of 3 μm and higher. The thinner sections in particular showed a considerable upward deviation from the preset value on the microtome.

The section thickness has a considerable influence on the intensity of the staining, as specimens with a higher section thickness often exhibit more intense staining or the staining process itself can be influenced by the section thickness. A strong deviation from the set section thickness is therefore particularly problematic for the subsequent staining, evaluation of the staining and the histopathologist's findings based thereon.
(iii) The sectioned specimen, which is placed on a slide, is then stained with various dyes. Staining of these sections is necessary for evaluation by the histopathologist, as the unstained components of the cells and tissue are difficult to distinguish under the optical microscope. The aim of staining is therefore to create a visual distinction between the components of the tissue. The standard method is double staining with hematoxylin and eosin, which stains the basic components of the cell (cell nucleus and cytoplasm). For special structures, such as connective tissue fibers, additional special stains (e.g. Goldner stain) are used. The introduction of immunohistochemistry was also a fundamental step forward. In this method, antibodies bind specifically to target proteins selected for examination. These specifically bound antibodies can be visualized on the microscope by means of a detection system that binds to the antibodies using dyes in the visible wavelength range or fluorescent dyes.
(iv) The subsequent evaluation of the stained histological specimen section is dependent on the experience of the evaluating histopathologist, who performs a subjective evaluation of the specimens. As a rule, the histopathologist assesses the morphological appearance of the sample, the presence, localization and staining of certain structures and the intensity of the staining. The staining intensity is usually assessed qualitatively or semi-quantitatively by estimating and classifying the staining intensity as “strong”, “medium”, “low” or “negative” and the distribution of the stained structures in the specimen section.

Only for individual diseases and associated target structures and special proteins has a finer grading been carried out to date using more detailed scores. This is done according to a fixed evaluation system, in which different individual criteria are often combined to form a score. Examples of this are the immunoreactive score 0-12 for estrogen receptors and progesterone receptors in breast cancer, scores for Her2neu, the proliferation-associated antigen Ki 67, the protein c-Myc, PD-L1 and others. More precise quantification of these factors is so important because a prognosis group and therapeutic decisions are derived from them. There is therefore a need for precise and objective quantification, particularly for such target structures and substances. However, these scores are also based on the subjective assessment and experience of the histopathologist. Quantitative measurement of a substance concentration in the histological specimen is not yet possible in everyday clinical practice, but is generally of great interest in order to enable a more precise assessment and diagnosis as a basis for therapy decisions, for example.

For the quantitative determination of substance concentrations in a clinical environment, it has previously been necessary to use clinical chemistry methods. In contrast to histology, clinical chemistry offers the possibility of quantifying target substances in biological material using highly precise methods. This mainly involves analyzing liquid samples (blood, blood serum and other body fluids). For specific protein determination, an immunoassay is usually carried out. The protein is bound with an antibody specific to this protein. This antibody can be coupled with an enzyme (ELISA), which produces color from a substrate. The density of the color can be measured photometrically and thus a concentration of the bound protein can be assigned. However, it is not possible in this case to localize the substance in the tissue, but only to determine an average value of the concentration of the target substance in the biological sample.

While the steps of embedding and staining can now be largely automated, especially in the clinical environment, and are therefore subject to minor fluctuations, it is clear from the above points that the steps of cutting the sample on the microtome and the assessment of the sample by the histopathologist in particular have not yet been automated and standardized and make precise and quantitative assessment of histological specimen sections difficult.

Various approaches have been investigated to enable a more objective assessment of histological specimens. In particular, various approaches to standardization have already been undertaken with regard to the assessment of images of histological specimens and the determination of target substances in histological specimens.

In recent years, it has become possible to scan histological specimen sections by combining special microscope optics with an image sensor and to create a high-resolution image of the entire specimen from a large number of individual images. This so-called slide scan can be used instead of viewing the specimen section under the microscope and is gradually being used in routine histopathology. However, histopathologists have previously also subjectively evaluated slide scans by assigning scores. Evaluation of the scan using image analysis and algorithms for image measurement with the help of artificial intelligence (AI), among other things, is under development and is already being used in some cases, although the focus is still on evaluating only relative color differences.

Nguyen et al., 2013 discloses the determination of relative staining intensity by measuring the optical density and then subtracting the intensity of the stained region from the maximum intensity.

Choudhury et al., 2009 discloses the use of an ATM score (Average Threshold Measure) to automatically set threshold values for positive staining in immunohistochemistry. This also demonstrated good repeatability and comparability, but does not allow absolute measurement of protein concentration.

Jensen et al., 2017 discloses an immunohistochemistry method for determining the Her2neu protein based on an amplification system that enables the determination of a protein in fixed and paraffin-embedded tissue by counting points in an image analysis. However, the method cannot be used to measure individual components of the cell (in particular cytoplasm and cell nucleus). The result is also presented as a histoscore. The so-called histoscore is based on the staining density and the percentage of stained cells and consists of four categories: negative (0), weak (1+), moderate (2+) and strong (3+).

Quantitative methods for determining an absolute substrate concentration in a histological specimen are disclosed in US 2004/0253649 A1 and U.S. Pat. No. 5,610,022.

US 2004/0253649 A1 discloses a method for quantifying cellular proteins in tissue samples using a cell imaging densitometer in conjunction with immunohistochemical staining and a reference standard that is stained in the same way as the actual tissue sample. The method enables the quantification of a target protein in terms of the absolute amount of the protein or the number of protein molecules per cell/volume/group in a tissue sample expressing the target protein.

U.S. Pat. No. 5,610,022 also discloses an internal control that can be used for quantifying substance concentrations in tissue samples by immunohistochemistry. The control is treated under the same conditions as the sample, including immunostaining of the tissue sample. The immunoreactivity of the control and the sample are compared by measurement before and after processing.

The aforementioned methods, in particular those described in US 2004/0253649 A1 and U.S. Pat. No. 5,610,022, enable a more precise, semi-quantitative or quantitative determination of substance concentrations in histological specimens. However, the large fluctuations in the thickness of the section during manual cutting on the microtome with fluctuations of up to 200% in the actual section thickness are not taken into account, but have a considerable influence on the color intensity of the staining, which forms the basis for determining the substance concentration in the respective methods. The fluctuations in the actual section thickness therefore significantly affect the accuracy and precision of the respective methods.

EP 1200799 B1 discloses a method and apparatus for determining the thickness of thin films based on ellipsometric measurement of the rotation of polarized light reflected by the thin film.

US 2013/0314712 A1 also discloses an apparatus and a method for determining the optical properties of a “thin film”, in which the “thin film” is simultaneously irradiated with light of different wavelengths and interference effects are measured. Reflection and transmission coefficients are then calculated and compared with calibration values. Based on this, the thickness and concentration of analytes in the thin film can be determined in an image of the thin film by a grayscale value analysis of the pixels of an image of the thin film and comparison with calibration or reference values. However, a specific method for determining the concentration or a specific analysis of selected regions of the thin film is not disclosed. Matenaers et al. (PLoS ONE 13 (2), 2018, p-1-21; e0192879) discloses the determination of the section thickness of a histological specimen using a reflectometer (measurement of spectral reflectance as a function of wavelength) as an alternative to the orthogonal re-embedding technique (ORE) commonly used in histology. However, the determination of section thickness by reflection is only described for hard sections such as synthetic resin and is not disclosed for paraffin sections. However, paraffin sections make up the majority of sections produced in histology. At the same time, in contrast to synthetic resin, for example, paraffin sections exhibit a low degree of homogeneity, which can sometimes severely impair the determination of section thickness by means of reflection. The applicability of this method to paraffin sections is therefore questionable.

Overall, all of these methods for measuring section thickness are based either on measuring only the surface or reflection and transmission. None of the methods investigates or takes into account special properties of paraffin sections, whose structure is more complex than, for example, the structure of hard sections such as synthetic resin. Due to this complex structure, a plurality of optical effects can overlap and considerably complicate and impair the determination of the section thickness using the known optical methods.

US 2006/0154234 A1 describes standardization and quantification methods for histology, immunohistochemistry and cytology. A reference standard for cytology and histology is disclosed which comprises an embedding medium and a compact particle such as a cell or a non-biological particle with cell-like dimensions. Here, the cell or particle contains a known amount of a substance (for example a protein or nucleic acid) which is to be quantitatively determined in the actual sample. However, the actual section thickness of the histological specimens is not examined when determining the substance concentration, and individual images of the section are used to determine the concentration, which can exhibit inhomogeneity in their illumination and thus impair the accuracy of the concentration determination. In addition, as explained above, only defined particles are measured and not the entire or an arbitrarily large region of the section. In order to enable an accurate and precise determination of substance concentrations in combination with the localization of the substance in histological specimens and thus a sound basis for diagnosis and therapy decisions based on these specimens, there is therefore a need to further develop and automate the histology method. This further development is particularly necessary with regard to the steps of the method, which are subject to strong local fluctuations, such as the actual section thickness of the specimens to be examined.

### SUMMARY OF THE INVENTION

The objective of the present invention was to eliminate the disadvantages of the prior art and to provide a method that enables the accurate and precise determination of substance concentrations and the localization of substances in specimen sections. One objective of the present invention was thereby to provide a method that determines the actual section thickness of a specimen section and a calibration specimen. A further objective of the present invention was to provide a correction factor for the actual locally different section thickness of the specimen section, which can be used for the exact determination of a substance concentration in the specimen section. Furthermore, another objective of the present invention was to provide a calibration specimen which contains the target substance to be determined and can be used for quantifying the substance concentration in the specimen section. In addition, another objective of the present invention was to provide an apparatus for determining the section thickness of a specimen section. Furthermore, another objective of the present invention was to provide a slide which can be used in the method according to the invention and in the apparatus and on which both the specimen section and the calibration specimen can be applied and simultaneously stained.

The objective according to the invention is solved by the features of the independent claims. Advantageous embodiments of the invention are described in the dependent claims.

In one aspect, the invention relates to a method for determining a section thickness of a specimen section, comprising (a) measuring one or more values generated from the scattering and/or absorption of a light beam by the specimen section with one or more light sources of different wavelengths, (b) providing modeling of the section thickness of the specimen section based on calibration values generated by measurement of the scattering and/or absorption of a light beam by one or more calibration specimens with one or more light sources of different wavelengths, and (c) determining the section thickness of the specimen section, generated by measurement of the scattering and/or absorption of a light beam by one or more calibration specimens having one or more light sources of different wavelengths, and (c) determining the section thickness of the specimen section by using the measured values according to (a) and modeling according to (b).

One major advantage of the method according to the invention is that it enables simple and precise determination of the actual section thickness of a specimen section. Specimen sections are usually prepared by cutting on the microtome. The actual section thickness can deviate considerably from the set section thickness, especially at low set section thicknesses. The section thickness can have a significant influence on the subsequent staining and evaluation by the histopathologist or the quantification of target substances in the specimen section. Conventional methods for determining the section thickness of a specimen section include complex and time-consuming microscopic methods such as the use of a laser scanning microscope. It is therefore a particular advantage of the method according to the invention that by measuring the scattering and/or absorption of a specimen section, the actual section thickness of a specimen section can be determined quickly and without the use of special microscopes or special knowledge on the part of the user. Providing a model of the section thickness by using calibration specimens and calibration values based thereon enables a broad application of the method, since the actual section thickness of any given specimen section can be determined directly based on the modeling and the measured values. Furthermore, in contrast to microscopic methods, the method enables a high-throughput determination of the actual section thickness of specimen sections, which makes it particularly advantageous for use in everyday clinical practice, where a high volume of specimen sections is produced.

Measuring scattering and/or absorption proves to be particularly advantageous for the method according to the invention, since the measured scattering and/or absorption values exhibit a high degree of sensitivity in relation to the section thickness. By measuring the scattering and/or absorption, in contrast to other optical effects such as in some cases transmission and in particular reflection, it is possible to determine the actual section thickness of the specimen section precisely and correctly over a wide wavelength range and thus also to increase precision and accuracy when evaluating specimen sections and determining the concentration of target substances in these specimens. In particular, measurement of scattering and/or absorption also makes it possible to measure the section thickness of section preparations which have an irregular structure and surface, such as paraffin sections, which represent a large proportion of the section preparations used in histology. For example, measuring transmission alone is not suitable for determining the section thickness of such specimens with an irregular structure, as a plurality of optical effects are superimposed to varying degrees, which means that it is not possible to establish a continuous correlation between section thickness and transmission.

In a further embodiment, the method is characterized in that the light source(s) exhibit a wavelength of 200 to 2000 nm, preferably 300 to 1800 nm, particularly preferably 400 to 1700 nm. In a further preferred embodiment, the light source(s) may have a wavelength of 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000 nm or other wavelengths in this range which achieve the desired properties of the present invention.

In a preferred embodiment, the method is characterized in that the modeling of the section thickness comprises (a) providing the calibration values that were determined by measuring the scattering and/or absorption of a light beam by one or more calibration specimens with one or more light sources of different wavelengths, wherein the calibration specimens exhibit a known section thickness of 0.1 to 10 μm, preferably a known section thickness of 1 to 7 μm, (b) determining one or more calibration functions, wherein the calibration function comprises correlating the section thickness of the one or more calibration specimens with the calibration values, (c) comparing the measured values of a specimen section with the one or more calibration functions according to (b), preferably taking into account a measurement value inaccuracy of 5%, (d) outputting a section thickness of the specimen section which corresponds to the mean value of one or more values which correspond to one or more of the calibration functions according to (c). In a further preferred embodiment, the light source(s) may have a wavelength of 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000 nm or other wavelengths in this range which achieve the desired properties of the present invention.

By providing modeling of the actual section thickness of a specimen section, a simple and broad application of the method according to the invention is possible. In contrast to microscopic methods for determining the actual section thickness, the method can be used without special knowledge and with easy-to-operate apparatuses, such as the apparatus according to the invention, and enables precise and accurate determination of the actual section thickness and its local distribution. Furthermore, the use of modeling in the method according to the invention enables automated and high-throughput determination of the section thickness, which can also be used by personnel without special knowledge, particularly in everyday clinical practice.

Advantageously, the calibration function can be used to determine a correction factor for a plurality of locally separate regions of the specimen section, which enables locally different corrections and, for example, the creation of a height profile of the specimen section as described in detail below.

In a further embodiment, the method is characterized in that the specimen section exhibits a section thickness of 0.1 to 10 μm, preferably 1 to 7 μm. In a further embodiment, the specimen section can have a section thickness of 0.1 μm, 0.2 μm, 0.3 μm, 0.5 μm, 0.6 μm, 0.7 μm, 0.8 μm, 0.9 μm, 1 μm, 1.5 μm, 2 μm, 2.5 μm, 3 μm, 3.5 μm, 4 μm, 4.5 μm, 5 μm, 5.5 μm, 6 μm, 6.5 μm, 7 μm, 7.5 μm, 8 μm, 8.5 μm, 9 μm, 9.5 μm, 10 μm or any other thickness in this range which achieves the desired properties of the present invention.

The method of the present invention can advantageously be used for the entire range of specimen section thicknesses commonly used in histology. Surprisingly, the method according to the invention exhibits a high degree of sensitivity over the entire range and enables accurate and precise determination of the actual section thickness. Surprisingly, the method also enables precise determination of the section thickness in the section thickness range of 1 to 2 μm, which is common in histology and for which a particularly high deviation from the section thickness set on the microtome has been observed, and thus precise quantification of target substances in sections of this thickness. Thus, the method according to the invention can be widely used, in particular in histology, and thus enable the precise evaluation and quantification of target substances and diagnoses and therapy decisions based thereon.

In a further embodiment, the method is characterized in that the specimen section contains tissue.

In a further embodiment, the method additionally comprises determining the concentration of a target substance in the tissue contained in a specimen section.

In a further embodiment, the method is characterized in that the determination of the concentration of the target substance comprises: (a) providing a specimen section and a calibration specimen, wherein the calibration specimen comprises a predetermined concentration of the target substance, (b) determining the section thickness of the specimen section and the calibration specimen, (c) determining a correction factor by using the section thickness according to (b), (d) simultaneously staining the specimen section and the calibration specimen, preferably on the surface of a slide, (e) measuring the color intensity of the tissue contained in the specimen section and the target substance contained in the calibration specimen, and (f) determining the concentration of the target substance in the tissue by using the color intensity according to (e) and the correction factor according to (c). In a further embodiment, the tissue-free part of the specimen section and/or the calibration specimen which contains a dye which absorbs light in a wavelength range from 200 to 2000 nm is covered or separated before staining.

In a further embodiment, the section thickness is only determined after the simultaneous staining of the specimen section. In a further embodiment, the locally different section thickness is only determined after the simultaneous staining of the specimen section.

In a further embodiment, the method is characterized in that the determination of the concentration of the target substance comprises: (a) providing a specimen section and a calibration specimen, wherein the calibration specimen comprises a predetermined concentration of the target substance, and the specimen section and the calibration specimen comprise one or more regions containing a dye which absorbs light in a wavelength range from 200 to 2000 nm, (b) covering or separating the regions of the specimen section and the calibration specimen which contain the dye, (c) simultaneously staining the specimen section and the calibration specimen, preferably on the surface of a slide, (d) determining the section thickness of the specimen section and the calibration specimen, preferably on the surface of a slide, (d) determining the section thickness of the specimen section and the calibration specimen by a method according to one of the previous claims, (e) determining a correction factor by using the section thickness according to (d), (f) measuring the color intensity of the tissue contained in the specimen section and the target substance contained in the calibration specimen, and (g) determining the concentration of the target substance in the tissue by using the color intensity according to (f) and the correction factor according to (e).

The method according to the present invention can advantageously be used to absolutely quantify a target substance in a specimen section. Here the use of a calibration specimen, which contains a known concentration of the target substance and is stained and measured simultaneously with the specimen section in the method according to the invention, makes it possible to determine the absolute concentration of the target substance. In combination with the determination of the actual section thickness of the specimen section and of the calibration specimen, which may differ locally, a correction factor (for example as a correction factor area based on a height profile, also called correction area) is provided, which considerably increases the accuracy and correctness when determining the concentration. In the methods used to date in the prior art to determine the absolute concentration of a target substance in histological specimens, the actual section thickness of the specimen sections is not determined and there is no calculation of a correction factor based thereon. However, since the section thickness of the specimen sections, such as histological specimens, can vary considerably, for example due to the sectioning method (e.g. using a microtome) or due to the embedding material used (e.g. due to local differences in the section thickness that occur in the case of paraffin-embedding), and can thus differ considerably within the specimen section or also between the specimen section and the calibration specimen, and at the same time has a significant influence on the intensity of the staining and thus the determination of the concentration of a target substance, the identification of a correction factor for the section thickness in the method according to the invention achieves a significantly higher degree of accuracy and precision for the determination of the absolute concentration compared to the prior art. A correction of these local differences in the section thickness, as they occur in the case of paraffin-embedding, and this being taken into consideration when determining the concentration, for example by creating a height profile as described below, has not yet been disclosed in the prior art.

With the method according to the invention, it is thus possible for the first time to carry out a precise absolute quantitative measurement of the concentration of a target substance with simultaneous localization of the target substance in a histological specimen section. Substances, in particular proteins, for which other quantitative measurement methods are available, can be objectively quantified by creating a calibration curve using the calibration specimen in comparison with the specimen section. The method according to the invention thus exhibits both the high quantitative accuracy of clinical chemistry and the high spatial resolution of histology. With the method according to the invention, localized and exact measurements of concentrations of a target substance are possible in individual cells and even in individual cell components (cell membrane, cytoplasm, cell nucleus, parts of the cytoplasm or the cell nucleus). This opens up new possibilities in medical diagnostics and research in particular. By using the method according to the invention, limit values can be determined for diagnostic decisions/therapy decisions and, in research, a high-resolution and absolute quantitative analysis of pathological processes is also made possible.

This method can be particularly relevant for diseases with a heterogeneous appearance, such as prostate carcinoma, in which the prostate-specific antigen (PSA) is formed. PSA can be detected immunohistochemically in the carcinoma and in the blood (serum) using clinical chemistry methods. Elevated blood values thereby indicate an increased probability of the presence of a carcinoma or metastasis, but can also be elevated in the case of inflammation. Furthermore, the expression in an existing tumor is individually different, heterogeneous and currently not exactly quantifiable. By using the method according to the invention, PSA quantification and localization in the tumor tissue can be achieved and, based thereon, individual cut-off values can be determined for each patient, which can replace the cut-off currently determined statistically for all patients. This would allow prognosis and treatment decisions for this tumor disease to be considerably more precise and individualized. The method can also be used in a similar way for other tumor diseases and markers, for example for the protein S100, which is expressed in malignant melanomas.

In a further embodiment, the method is characterized in that the calibration specimen contains the target substance in two or more concentrations which are spatially separated from each other.

In a further embodiment, the method is characterized in that the section thickness of the specimen section and of the calibration specimen is determined in tissue-free areas of the specimen section and the calibration specimen.

In a further embodiment, the method is characterized in that the values generated from the scattering and/or absorption of a light beam by the specimen section and/or the calibration specimen are measured at one or more positions in the tissue-free regions of the specimen, preferably at two or more positions, particularly preferably at two or four positions. In a further embodiment, the method is characterized in that the values generated from the scattering and/or absorption of a light beam by the specimen section and/or calibration specimen are measured at two or more positions in the tissue-free regions of the preparation which are arranged symmetrically around the tissue contained in the specimen section, preferably at opposite tissue-free regions. In a preferred embodiment, the method is characterized in that a mean value is formed from the two or more positions measured, which is used to determine the section thickness of the specimen section. In a further embodiment, a two-dimensional function (correction area) is determined. Such a correction area takes into account any local or localized differences in the slice thickness in the specimen section and can therefore entail locally different correction factors.

In a further embodiment, the method is characterized in that the tissue-free regions of the specimen section, of the one or more calibration specimens comprise one or more regions which contain a dye which absorbs light in a wavelength range from 200 to 2000 nm, for example 200 nm, 250 nm, 300 nm, 350 nm, 400 nm, 450 nm, 500 nm, 550 nm, 600 nm, 650 nm, 700 nm, 800 nm, 850 nm, 900 nm, 950 nm, 1000 nm, 1050 nm, 1100 nm, 1150 nm, 1200 nm, 1250 nm, 1300 nm, 1350 nm, 1400 nm, 1450 nm, 1500 nm, 1550 nm, 1600 nm, 1650 nm, 1700 nm, 1750 nm, 1800 nm, 1850 nm, 1900 nm, 1950 nm or 2000 nm, preferably in a range from 300 to 800 nm,

In a further embodiment, the method is characterized in that the section preparation, the one or more calibration specimens and the calibration specimen are a paraffin section.

In a further embodiment, the method is characterized in that the specimen section and/or the calibration specimen is obtained by embedding tissue and/or one or more calibration cones containing prepared tissue of a known concentration in a paraffin block which is subsequently cut. In a further embodiment, the method is characterized in that the specimen section is obtained by embedding tissue in a paraffin block (sample block) which is subsequently sectioned. In a further embodiment, the method is characterized in that the calibration specimen is obtained by embedding one or more calibration cones containing prepared tissue of a known concentration in a paraffin block (calibration block), which is subsequently cut. In a further embodiment, the method is characterized in that the calibration specimen is obtained by injecting a solution containing a defined concentration of the target protein into one or more already prepared calibration cones, which are subsequently embedded in paraffin and cut. In a further embodiment, after injection of a solution containing a defined concentration of the target protein, the calibration cone is stored in formalin to solidify the protein. In a further embodiment, the target protein is a protein isolated from a tissue and/or a recombinantly produced protein.

In a further embodiment, the method is characterized in that one or more dyes which absorb light in a wavelength range from 200 to 2000 nm are introduced into the tissue-free region of the sample block and/or the calibration block and the sample block and/or the calibration block are subsequently cut to obtain a specimen section and/or a calibration specimen. In a further embodiment, the method is characterized in that the dye is introduced into the sample block and/or calibration block by introducing one or more punches (calibration punches). In a further embodiment, the method is characterized in that the dye is introduced into a separate paraffin block, from which one or more samples are removed by punching, which are introduced into the sample block and/or the calibration block, preferably by punching.

In a further embodiment, the method is characterized in that the section thickness of a specimen section is determined by measuring values obtained from the absorption of a light beam by the specimen section in the one or more regions in which a dye has been introduced. In a further embodiment, the method is characterized in that the section thickness of a specimen section is determined by measuring values obtained from the absorption of a light beam by the specimen section in the one or more areas in which the calibration punch has been introduced.

In a further embodiment, the method is characterized in that the section thickness of a specimen section is determined by measuring values obtained from the absorption of a light beam by the specimen section in the two or more regions in which a dye has been introduced. In a further embodiment, the method is characterized in that a profile of the section thickness (height profile) of the paraffin-embedded section is created based on the determination of the section thickness of a specimen section by measuring values determined from the absorption of a light beam by the specimen section in the two or more regions in which a dye has been introduced.

In one embodiment, the creation of the height profile comprises a two-dimensional fitting function. In a further embodiment, the two-dimensional fitting function comprises section thicknesses or equivalent section thicknesses (interpolation points) determined at two or more different positions of the specimen section. In a further embodiment, the creation of the height profile additionally comprises interpolation from the interpolation points by means of applying the two-dimensional fitting function to the entire specimen section.

In a further embodiment, a correction factor is determined for each region of the specimen section and/or the calibration specimen (control region) using the height profile, and the concentration of a target substance in a region of the specimen section is determined using the correction factor for this region. In a further embodiment, the concentration of a target substance in a region of the specimen section is determined using a correction factor for this region, wherein the correction factor is a constant correction value for the entire specimen section or a locally resolved correction factor (correction area created by means of a height profile).

In a further embodiment, the method is characterized in that a digital scan is made of the specimen section and/or the calibration specimen, which is used for measuring the values and/or the color intensity, wherein the measurement of the values and/or the color intensity comprises the determination of a brightness value. Creating a digital scan in the method according to the invention enables the standardized and also automated creation of calibration curves based on calibration specimens and evaluation of the specimen section and determination of the concentration of a target substance. Simply photographing a specimen section or calibration specimen entails considerable fluctuations in image quality due to variable magnification, vignetting of the image field and different lamp brightness, color temperature and exposure time. In contrast, creating a digital scan generates an image that exhibits a uniform brightness with the same white balance and as a result of calibration can always be carried out in the same way, even with different specimen sections. The generated image can then be standardized and automatically evaluated and offset against the correction factor or the plurality of correction factors (correction area) of the actual section thickness determined by means of the height profile to determine the section thickness of a specimen section and used to determine the concentration. Conventional photo processing programs known to the person skilled in the art can also be used for the evaluation.

In contrast to the method disclosed in US 2013/0314712 A1, in which the section is only measured as a whole or in certain regions in the apparatus, but not on the scanned image, a digital scan of the specimen section according to the present invention enables a pixel-precise resolution of the specimen section. On a digitally scanned image, which is composed of numerous individual images, a uniform image brightness/base intensity of the signal is guaranteed over the entire specimen, since optically generated images always exhibit a more or less pronounced edge darkening.

In a further embodiment, a mathematical program is used to determine the correction area. In a further embodiment, the mathematical program is used to generate a false color diagram with a color key, wherein the local concentrations of the target substance are encoded in the color key.

Advantageously, such a mathematical program thus provides information about the local concentrations of the target substance in a specimen section.

In a further embodiment, the method is characterized in that the measured color intensity of the target substance present in the calibration specimen is used to create a calibration curve for the concentration of the target substance, which is preferably determined from two or more concentrations of the target substance.

By providing two or more concentrations of the target substance, it is possible to create a calibration curve that can be used to determine the concentration accurately and precisely. The spatial separation of the concentrations in the calibration specimen avoids overlapping and mixing of the concentrations and thus avoids incorrect determinations of the concentration. The spatial separation and provision of different concentrations can be achieved very easily using known techniques.

In a further embodiment, (a) the specimen or tissue containing the target substance is prepared according to methods known in the prior art, preferably by mechanical comminution and/or dissolution of the tissue in a lysis buffer, (b) the concentration of the target substance is determined preferably by the methods of clinical chemistry, (c) the prepared specimen is diluted so that it contains a specific concentration of the target substance and (d) the prepared specimen or tissue according to (c) is bound in a gel in the form of a calibration cone (cell block). In a further embodiment, one or more of the calibration cones are introduced into a paraffin block (calibration block) spatially separated from one another. In a further embodiment, this calibration block can be used in the method according to the invention for the production of a calibration specimen, preferably by producing a section of the calibration block.

The calibration blocks according to the present invention can advantageously be produced using standardized methods for any target substances such as proteins and made available to the user of the method. As a result, a high degree of global standardization of the method according to the invention can be achieved.

In a preferred embodiment, the method is characterized in that the target substance is a substance within the tissue contained in a specimen section, preferably a protein, and the target substance is localized in the tissue contained in the specimen section and the concentration of the target substance is determined, based on the calibration specimen and the calibration curve.

In a further aspect, the invention relates to an apparatus for measuring the section thickness of a specimen section and optionally for determining the concentration of a target substance in the specimen section, comprising (a) one or more light sources of different wavelengths in the range from 200 to 2000 nm, preferably 300 to 1800 nm, particularly preferably 400 to 1700 nm, (b) at least one sensor for measuring the scattering and/or absorption of a light beam, (c) an apparatus for positioning the specimen section during the measurement, and (d) a computing unit for modeling the section thickness of a specimen section or for using the modeling in determining the section thickness of a specimen section. In a further preferred embodiment, the one or more light sources may have a wavelength of 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 or 2000 nm or other wavelengths in this range which achieve the desired properties of the present invention.

In a further embodiment, the apparatus is characterized in that the apparatus comprises a shaping structure into which one or more light sources and one or more sensors can be inserted, preferably in opposite positions. In a further embodiment, the apparatus is characterized in that a slide on which a specimen section and/or a calibration specimen is applied can be inserted between the light source and the sensor and the light beam emitted by the light source is directed through the slide and the scattering and/or absorption of the light beam can be measured by the sensor. In a further embodiment, the light source is a light-emitting diode or a laser diode. In a further embodiment, the sensor is a photodiode and/or a scanner. In a further embodiment, the apparatus is characterized in that the apparatus comprises one or more apertures which limit the light beam emitted by the light source to one or more specific positions of the slide on which a specimen section and/or a calibration specimen is applied. In a further embodiment, the apparatus comprises a camera which by means of the light source can record the screening position of the slide limited by the one or more apertures. In a further embodiment, the camera can be used for creating an image of the specimen section, for example for image evaluation and/or for determining further properties of the specimen section, for example for determining the homogeneity of the specimen in the form of measured values which can be used for determining the section thickness or a concentration of a target substance in a specimen section.

In a further aspect, the invention relates to a slide for use in the method and/or with the apparatus, comprising a calibration specimen containing a target substance in two or more concentrations which are spatially separated from each other, onto which the specimen section can additionally be applied. In a further embodiment, the slide comprises a coverable and/or separable region. In a further embodiment, the tissue-free part of the calibration specimen, containing a dye that absorbs light in a wavelength range from 200 to 2000 nm, is applied onto the separable region, preferably in the form of a calibration punch. In a further embodiment, the tissue-free part of the specimen section, which contains a dye that absorbs light in a wavelength range from 200 to 2000 nm, is applied onto the separable region, preferably in the form of a calibration punch.

By providing a slide onto which the specimen section and the calibration specimen can be applied, simultaneous sample processing is made possible. Simultaneous sample processing means that the method according to the invention can be carried out within a short time, since the sample preparation steps only have to be carried out once and not separately for the specimen section and the calibration specimen. Furthermore, the method according to the invention is further standardized by the simultaneous sample preparation, since any fluctuations that may arise as a result of separate sample processing are eliminated. The provision of a slide for the method according to the invention further facilitates its applicability, for example, in a clinical environment, since only the specimen section has to be applied and the standardized steps of the method have to be carried out. By covering or separating the calibration punches, disintegration of the calibration punches can advantageously be avoided, making it possible to determine the section thickness after staining the specimens in one step, for example by a scan of the specimens, which can then be used to determine the section thickness, the calibration curve and the concentration of the target substance. If the calibration punches are not covered, the section thickness must be determined before staining, as the calibration punches disintegrate during staining and it is therefore no longer possible to determine the section thickness subsequently.

The aforementioned features relating to the method for determining the section thickness of a specimen section have structural and functional implications for the other aspects of the invention which comprise the method for determining the concentration of a target substance in the tissue contained in a specimen section, the apparatus for measuring the section thickness of a specimen section and the slide for use in the method according to the invention, such that the method for determining the section thickness, the method for determining the concentration of a target substance, the apparatus for measuring the section thickness and the slide can each be described by or derived from the features of the other aspects.

### DETAILED SUMMARY OF THE INVENTION

All cited documents of patent literature and non-patent literature are hereby incorporated by reference in their entirety. Each term described below is to be understood in its general meaning as known to those skilled in the art. The definitions are provided below by way of example to illustrate how each term is applied in the context of the invention and may be used in preferred embodiments of the invention.

Histology and histopathology involve the microscopic examination of biological material such as tissue samples in terms of morphology. In histopathology, the sample to be examined under the microscope is usually biological material in the form of a tissue sample taken during an operation or medical examination, also known as a biopsy. A biopsy is a procedure to remove and obtain a small amount of tissue from a person or patient. The different types of biopsy include punch biopsy, needle biopsy, endoscopic biopsy, excisional biopsy and perioperative biopsy. Larger samples consist of organs or parts of organs (“resectates”) and also require the removal of representative material by the pathologist before further examination. The person skilled in the art is familiar with these procedures. Typical samples obtained by biopsy include tissue from the colon, kidney, pancreas, cervix, lung, breast, prostate or soft tissue.

For histological or pathohistological examination, the removed sample is then (i) fixed, (ii) clarified/embedded, (iii) sectioned and (iv) stained. Alternatively, frozen sections can be prepared using a cryostat if rapid results are required or if fixation would compromise the target structures. Fixation of the sample is carried out to prevent autolysis and decay and to preserve structures present in the tissue during the subsequent steps. Tissue samples can be fixed using physical or chemical methods. Physical methods comprise heat treatment and microwave treatment as well as cryopreservation. Chemical fixation is usually carried out by immersing the specimen in a fixative. In addition, there are fixatives in the form of vapor used for some special histochemical procedures, such as vapor fixation of freeze-dried tissues using paraformaldehyde and osmium tetroxide. Chemical fixatives comprise alcohols, aldehydes such as formalin, neutral buffered formalin (4-10% formaldehyde in isotonic buffered saline), paraformaldehyde, glutaraldehyde and acrolein, oxidizing agents such as potassium permanganate, osmium tetroxide and chromic acid and tanning agents. Biological tissue samples are often transferred to a fixative, usually 10% neutral buffered formalin, for 24 to 48 hours immediately after removal.

For the subsequent dehydration, the sample is immersed in increasing concentrations of alcohol to remove water and formalin from the sample. Dehydration is followed by clarification with an organic solvent such as xylene to remove the alcohol and allow infiltration and thus embedding in paraffin wax. During embedding, the sample is infiltrated with the embedding agent-usually paraffin. The embedding agent forms a carrier matrix and subsequently allows the sample to be cut into very thin sections that can be examined microscopically.

A section, also known as a “specimen section”, is usually made using a microtome. A microtome is a cutting device with which very thin sections can be obtained with a section thickness in the range of 0.1 to 100 μm. The term “section thickness” or “section thickness” describes the diameter or the height of the specimen section, which can be uniform (constant height over the area) or variable (locally variable). A microtome usually consists of a knife block, a specimen holder and a feed mechanism. During cutting, the knife is pushed through the specimen and a thin section is obtained, which is pushed out of the device by the feed mechanism to allow the next section to be produced. Various types of microtomes are known, such as sliding microtomes, rotary microtomes, vibratomes, saw microtomes and laser microtomes. A person skilled in the art can select from these devices a suitable device for producing specimen sections. For histological examinations, specimen sections are prepared with a section thickness of 1 to 10 μm, preferably 2 to 7 μm. The prepared sections are collected in a basin of water and stretched out on the surface of the water by the surface tension and can thus be transferred to a slide for further sample preparation.

The term “paraffin-embedded section” refers to a specimen section obtained by cutting a paraffin block. A histologic sample containing tissue may be embedded in this paraffin block. The regions of the paraffin block in which no tissue is embedded are referred to as “tissue-free regions”. The embedding in a paraffin block serves to support the tissue structure, enables the preparation of very thin specimen sections of the tissue and the mounting of the specimen sections on a slide for subsequent examination, for example microscopic examination. The term “histological specimen section” refers to a specimen section containing tissue obtained, for example, by biopsy.

Paraffin is a mixture of saturated acyclic hydrocarbons with the general molecular formula CₙH₂ₙ₊₂, wherein the number n is between 18 and 32. Paraffin is mobile, oily or waxy, and inert to many chemicals. Paraffin is used in histology as an embedding agent for histological specimens. At a wavelength of 300 nm or higher, paraffin is highly translucent (Li et al. 2015). In the very short wavelength range (near 200 nm and below), paraffin becomes opaque even with a very thin section thickness.

The term “extinction” describes the reduction of radiation such as from a light beam, also known as a “light source”, when it passes through a medium such as a paraffin-embedded section. The extinction coefficient characterizes the strength of the interaction of the radiation with the medium. It is a material constant that depends on the wavelength of the incident radiation. Extinction is the overall effect of attenuation and is divided into the components reflection, absorption, scattering and diffraction. The attenuation of the intensity along the path through the medium is proportional to the absorption coefficient of the irradiated material and its thickness. This results in the Beer-Lambert law.

The Beer-Lambert law describes the attenuation of the intensity of radiation in relation to its initial intensity when passing through a medium containing an absorbent substance, as a function of the concentration of the absorbent substance and the section thickness of the medium. Absorbing substances can be, for example, paraffin, natural pigments contained in histological specimens such as melanin or dyes used to stain histological specimens. Absorbent substances can also be dyes or mixtures of dyes, which are introduced into the sample block or calibration block in the form of a calibration punch. Preferably, these dyes absorb in a wavelength range from 200 to 2000 nm, particularly preferably in the range from 300 to 800 nm. Dyes that absorb in this range include, for example, anthraquinones (e.g. alizarin and carmine), azo dyes (e.g. aniline yellow and Congo red), dioxazine dyes (e.g. Sirius light blue and carbazole violet), indigoid dyes (for example indigo and indirubin), formazan dyes (for example reactive blue 235), methine dyes (for example β-carotene, lycopene and basic red 12), nitro and nitroso dyes (for example acid orange 3 and acid green 1).

There are several variants of the notation of the Beer-Lambert law. One of them is:

$${E}_{λ} = {log}_{10} ( \frac{{I}_{0}}{{I}_{1}} ) = {ε}_{λ} ( λ ) c d$$

wherein I₀ is the intensity of the incident light beam, I₁ is the intensity of the transmitted light, ε_{λ} (λ) is the wavelength-dependent absorption coefficient or extinction coefficient, c is the mass concentration of the absorbing substance and d is the thickness of the medium through which the light flows. The Beer-Lambert law can be established for any wavelength λ in order to calculate the specific absorption of the light of this wavelength with the intensity I₀(λ), which gives the intensity of the transmitted light I₁(λ).

The term “reflection” describes the reflection of radiation when it hits the interface between two different media. Reflection is therefore a phenomenon that occurs on surfaces and is not dependent on the section thickness of a specimen section. The term “absorption” describes the absorption of radiation by a medium and is reciprocal to transmission, which describes the relative optical transmittance of a medium. Absorption depends on the wavelength of the radiation and the medium. The term “scattering”, also known as “light scattering”, describes the splitting of a light beam by irregularities in a translucent medium, such as atoms, molecules, bacteria or droplets in an emulsion or in mist. The scattered light intensity is distributed differently in the surrounding space depending on the type, shape and size of the scattering object. As a result, the radiation passing through a medium is attenuated. This attenuation of the radiation is measured together with the effects of absorption, reflection and diffraction as part of an extinction measurement of a medium. According to the present invention, the effects of reflection and diffraction when passing through a specimen section are negligible and the extinction measurement is substantially based on the measurement of scattering and/or absorption. Devices for measuring absorbance include, for example, turbidimeters, nephelometers, spectrometers such as UV/VIS spectrometers or infrared spectrometers and are known to a person skilled in the art. In the context of transmitted light measurements in liquids, the term turbidimetry is also used by a person skilled in the art, which is distinguished from nephelometry (scattered light measurement). In the present invention, the principles of turbidimetry are preferably used to determine the section thickness of a specimen section, while the principles of angle-dependent resolved nephelometry can preferably be used for further indications of the spatial distribution of concentrations (for example in the thickness direction of the section).

The term “light source” comprises a physical system that emits electromagnetic radiation from the X-ray to the infrared range, usually in a limited spectral region. Common light sources that can be used for extinction measurements such as scattering and/or absorption measurements comprise, without being limited to, continuum emitters such as halogen lamps and xenon lamps, line emitters such as cadmium and mercury lamps, light emitting diodes (LEDs), fluorescent tubes and lasers.

The term “calibration” comprises the measurement of a specimen or a sample of known property by means of a method or a measuring method in order to use this method to determine this property in a specimen with an unknown property. The property to be determined may include, for example, the concentration of a certain substance in the specimen or the section thickness of the specimen. The property to be determined may also comprise, for example, constants in the mathematical model for evaluating the measurement data. The specimen with the known property used for the calibration is referred to as the “calibration specimen” and the values measured using this calibration specimen are referred to as “calibration values”. Using these calibration values, properties such as the concentration of a particular substance can be determined and calculated from the values from a specimen with unknown properties obtained using the method.

The term “calibration curve”, also known as a “standard curve”, comprises a mathematical relationship or correlation of a plurality of determined calibration values, which were determined, for example, by measuring a plurality of known concentrations of a specimen or measuring the scattering and/or absorption of different specimens with known section thicknesses. This calibration curve can be used to determine the properties of a specimen with unknown properties using the method that was used to create the calibration curve. The calibration curve usually comprises measured values in the range to be expected for the sample or specimen with unknown properties.

For the purposes of the present invention, the term “modeling” comprises the mathematical representation (calculation) of the properties of a specimen with unknown property based on one or more calibration curves for this property. This involves providing the calibration values, generating one or more calibration curves, comparing the values measured for a specimen with unknown properties with the calibration curves, and calculating a corresponding value for the undisclosed property of that specimen based on the calibration curves. Furthermore, “modeling” can include the application of additional mathematical formulas that describe physical relationships, such as the Beer-Lambert law.

Based on the modeling, a correction factor for a sample can be calculated in the present invention. A correction factor is a variable introduced into a calculation that is necessary to compensate for the influence of a particular variable or effect that would otherwise distort or influence the calculation. In the present invention, a correction factor is calculated based on the actual section thickness of a specimen section. This correction factor for the section thickness is used in the calculation of the concentration of a target substance in the specimen to compensate for the influence of the section thickness on the color intensity of the staining on which the concentration calculation is based.

The term “histological staining” refers to the use of histochemical dyes to treat tissue sections (preferably embedded in paraffin or frozen). Histological staining may also include one or more additional intermediate steps, such as the addition of an enzyme. Immunohistochemistry is a special form of staining in which the target protein is labeled with an antibody in an intermediate step; a second antibody binds to the first and is coupled, for example, with fluorescent dye or an enzyme that secondarily produces the color from a special substrate. Commercial antibodies are available for a large number of proteins, which a person skilled in the art can select accordingly.

In the context of the present invention, the terms “histological staining”, “histochemical staining” and “immunohistochemical staining” are used synonymously. Specimen sections are usually stained for a histological examination in order to stain structural features of the tissue and to increase the tissue contrast between structural features. Specific target structures and target substances can be stained through targeted selection of the dye. Target substances relevant for histology comprise, without being limited to, proteins such as cell receptors (e.g. estrogen receptors, progesterone receptors, Her2neu and c-Myc) and other proteins occurring intra- and/or extracellularly (e.g. Ki67 and PSA) or other components such as nucleic acids, mucous substances and lipids. Typical histochemical staining comprises, for example, the staining of cytochrome c oxidase, ATPases and NADH.

Common histological stains comprise, without being limited to, staining with hematoxylin and eosin (HE stain), azo carmine and aniline blue (Azan stain), resorcinol, fuchsin and orcein (Elastica stain), azur, eosin and methylene blue (Giemsa stain), iron hematoxylin, picric acid and acid fuchsin (van Gieson stain), iron hematoxylin and light green (Masson-Goldner trichrome stain), Iron hematoxylin (staining according to Weigert), iron hematoxylin (according to Heidenhain, EH staining), silver ions (Golgi staining), copper phthalocyanine (Luxol Fast Blue staining), cresyl violet, toluidine blue or thionine (Nissl stain), hematoxylin according to Harris, Orange G and polychrome solution (Papanicolaou stain), periodic acid (PAS stain), Sirius red (Pikro-Sirius red stain) and silver (Wartin-Starry stain). Other dyes that can be used alone or in combination and in combination with other chemicals for histological staining comprise, without being limited to, alizarin, Victoria blue B, acid red 88, eosin, acid fuchsin, Congo red, Safranin, basic fuchsin, crystal violet, methylene blue, hematoxylin, nuclear red, ethyl green, DRAQ5™, DRAQ7™, Nuclear Yellow (Hoechst S769121), Nuclear Green DCS1, Hoechst, DAPI, propidium iodide, Acid Fast Bacteria stain, Alcian blue (pH 1.0), Alcian Blue (pH 2.5), Alizarin Red, Colloidal Iron, Hydroxystilbamidine, Berlin Blue, Oil Red O, Safranin O stain, Sudan Black B and Toluidine Blue.

The term “color intensity” also called “signal intensity” refers to the strength of the measurable output signal that reflects the concentration of the dye bound to any region of a tissue section, e.g. a cytoplasmic region of a cell, a nuclear region of a cell, an interstitial region or an empty region. The color intensity can be determined, for example, by means of generally known image processing programs as a brightness value or tonal value, which is the brightness value of an image pixel in a color channel.

The term “calibration specimen” or “reference specimen” refers to a specimen section that contains a known concentration of a target substance and can be used to generate a calibration curve that correlates, for example, a measured color intensity with the known concentration of the target substance. According to the present invention, calibration specimens can be prepared from tissues, cells or substances that verifiably contain the target substance. The concentration of the target substance can be determined, for example, by methods commonly used in clinical chemistry, such as ELISA or mass spectrometry. Based on the known substance concentration, various concentrations in the appropriate concentration range can be produced by dilution.

Clinical chemistry uses only liquid samples as standard. According to the present invention, the liquefied tissue is solidified and used as a reference specimen (calibration specimen). The calibration specimens are produced in the form of cell blocks, also known as “calibration cones”, in which the tissue, cells or substances are bound in a defined concentration in a gel. One or more of these cell blocks of different concentrations can then be embedded in a matrix such as paraffin, in a so-called calibration block. By embedding them in an array, the cell blocks are spatially separated in different concentrations in the calibration block. A calibration block can also be produced by injecting a target protein in the form of a solution containing a defined concentration of the target protein into one or more calibration cones. The target protein can be a protein isolated from a tissue and/or a recombinantly produced protein. The protein is solidified by subsequently storing the calibration cone in formalin and embedding it in paraffin. A calibration block can be cut by means of a microtome and used as a calibration specimen in the present invention. One or more calibration punches containing absorbent substances such as dyes or mixtures of dyes may be incorporated into a calibration block. A calibration specimen produced from such a calibration block can thus contain calibration punches which contain absorbent substances such as dyes or mixtures of dyes.

The term “calibration specimen” refers to a specimen section which has a known section thickness, contains no tissue and can be used to create a calibration curve which correlates, for example, values measured from scattering and/or absorption with the known section thickness. Preferably, a calibration specimen exhibits a known film thickness of 0.1 to 10 μm. A calibration specimen can be produced from a calibration block which is cut using a microtome. In preferred embodiments, the calibration block is a paraffin block containing no tissue. The calibration block may contain one or more calibration punches which contain absorbent substances such as dyes or mixtures of dyes in a carrier material. A calibration specimen produced from such a calibration block can thus contain calibration punches which contain absorbent substances such as dyes or mixtures of dyes. Such a calibration punch may contain a carrier material which dissolves during the staining and/or fixation of the histological specimen section and/or contain material which does not dissolve during the staining and/or fixation of the histological specimen section.

A slide is an article on which objects and specimens such as specimen sections can be placed and viewed under a microscope. Materials used for slides include soda-lime glass, quartz glass or plastics such as polystyrene. Preferably, the slides of the present invention are made of quartz glass.

The term “sensor”, also known as a “detector”, comprises an apparatus that can detect certain physical or chemical properties (e.g. the brightness, extinction, absorption, scattering of a light beam) and/or the material properties of its surroundings qualitatively or as a measured variable qualitatively and/or quantitatively. These variables are detected by means of physical, chemical or biological effects and converted into a signal that can be further processed, such as an electrical signal or an image. According to the present invention, a sensor can be a light sensor such as a photodiode, a camera, or a scanner.

The term “digital scan” comprises the generation of an image of the specimen section or calibration specimen using a scanner. The image exhibits the same brightness with the same white balance in all sections and is recorded in the same way for all specimens using a standardized method. The digital scan generated can be used to analyze the brightness values or tonal values, for example by using an image processing program known to the person skilled in the art or for an automated evaluation.

In some embodiments, the apparatus according to the invention comprises a computing unit for modeling or for using the modeling. The computing unit is preferably a conventional computer device comprising a processor, an input device such as a keyboard or a mouse, a memory such as a hard disk and a volatile or non-volatile memory and a computer code (software). The computer may also include a programmable circuit board, microcontroller or other device for receiving and processing data signals as received from the local controllers, programmable manufacturing equipment, programmable material handling equipment and robotic manipulators. The apparatus may comprise a computing device preloaded with the necessary computer code or software that performs the modeling steps according to the invention. In certain embodiments, a plurality of clients such as desktop, laptop or tablet computers may be connected to a server such that, for example, a plurality of users may use the modeling according to the invention simultaneously. The computer system may also be networked with other computers via a local area network (LAN) or via an internet connection. The system may also comprise a backup system that stores a copy of the data obtained according to the invention.

The term “correction area” refers to a mathematical area function that is used to correct the height profile (locally different heights in the paraffin and in the embedded tissue). Correction in this context means that a uniform section thickness (or equivalent section thickness) is achieved at every location in the section (both in the calibration punches and in the regions of the specimen containing tissue). This enables the subsequent conversion of the locally determined color values (e.g. in a digital scan of the specimen section) into concentration values of the target substance.

The components of the computing device and of the computing code may be conventional, although the computing device and the computing code are individually configured for each specific implementation. The computing code can run on any architecture, such as personal computers/microcomputers, minicomputers, microcontrollers or mainframe systems. Examples of operating systems include Apple Mac OS X and iOS, Microsoft Windows and UNIX/Linux, SPARC-, POWER- and Itanium-based systems, and the z/Architecture. The computing code for carrying out the invention may be written in any programming language or model-based development environment, such as, but not limited to, C/C++, C#, Objective-C, Java, Basic/VisualBasic, MATLAB, Simulink, StateFlow, Lab View or Assembler. The computing code may include subroutines written in a proprietary computer language specific to the manufacturer of a circuit board, controller, or other computer hardware component used in connection with the invention.

In certain embodiments, the computing unit may contain amplification electronics for the output signal of a photodiode in the form of a transimpedance amplifier, which is realized in the form of an integrated circuit on a circuit board. In further embodiments, the measurement signal can be read out and processed using a microcontroller (e.g. Arduino). In further embodiments, the measurement signal can be processed in a microcontroller and then further processed in a connected computing device. In certain embodiments of the invention, a human supervisor may be present to monitor the modeling and correct any errors or malfunctions. However, in preferred embodiments of the automated or semi-automated embodiment of the method, the supervisor is not substantially involved in the method beyond preparing and attaching a slide to the microscopic equipment, and therefore need not routinely intervene in the computer-implemented aspects of the invention.

### REFERENCES

Masuda, Tomoyuki, et al. “How thick are the paraffin-embedded tissue sections routinely prepared in laboratory? A morphometric study using a confocal laser scanning microscope.” Pathology international 48.3 (1998): 179-183.
Nguyen, David. “Quantifying chromogen intensity in immunohistochemistry via reciprocal intensity.” (2013).
Choudhury, Kingshuk Roy, et al. “A robust automated measure of average antibody staining in immunohistochemistry images.” Journal of Histochemistry & Cytochemistry 58.2 (2010): 95-107.
Jensen, Kristian, et al. “A novel quantitative immunohistochemistry method for precise protein measurements directly in formalin-fixed, paraffin-embedded specimens: analytical performance measuring HER2.” Modern Pathology 30.2 (2017): 180-193.
Li, Dong, et al. “Optical properties of a liquid paraffin-filled double glazing unit” Energy and Buildings 108 (2015) 381-386.
Matenaers et al, Practicable methods for histological thickness measurement in quantitative stereological analyses, PLOS ONE 13 (2), 2018, p-1-21; e0192879.

### FIGURES

The invention will be explained in more detail below with reference to the figures and examples. The figures and examples do not limit the scope of the invention, but represent preferred embodiments of aspects of the invention for the purpose of illustration.

FIG. 1: Tissue with natural tissue pigment (melanin/iron) cut by microtome in different section thicknesses (A) 1 μm, (B) 3 μm, (C) 5 μm and (D) 7 μm.

FIG. 2: Transmission curve (transmittance) of liquid paraffin measured at different section thicknesses, depending on the wavelength (according to Li et al. 2015) (prior art).

FIG. 3: Measurement signal (in mV) after passage of UV-C (Δ=260 nm) through tissue-free paraffin-embedded sections as a function of the section thickness in μm (u) (average of a plurality of measurements), 0=blank value (glass only).

FIG. 4: Measurement of the scattering of a red laser (650 nm) through tissue-free paraffin-embedded sections as central brightness in the projection photo as a function of the section thickness in μm (μ), left blank value (section thickness 0, glass only), far left black value (darkest section of the photo without laser) as average values from a plurality of measurements (n≥6).

FIG. 5: (A) to (E) Comparison of the measurement curves (scattering) at different wavelengths, plotted against the section thicknesses 1, 3, 5 and 7 μm (μ) (blank value=0) of a tissue-free paraffin-embedded section: (A) infrared (direct measurement photo), (B) red (central brightness in projection photo), (C) blue (central brightness in projection photo), (D) violet (central brightness in projection photo) and (E) UV-C transmittance (sensor, in mV). (With the exception of the measurement in the UV-C range, the maximum is 3 μm. A comparison of 1 μm and 5 μm shows a shift of the maximum towards 1 μm in the infrared range and towards 5 μm in the violet wavelength range, illustrated by a straight line. The angle of inclination of the straight line is plotted in (F) against the wavelength and shows a clear monotonic relationship.

FIG. 6: Curves with associated fitting functions. TW denotes tonal values TWᵢ∈[0; 255] and U the measured voltage.

FIG. 7: Example measured values for the thickness of 5 μm. Highlighted are plausible thicknesses recognized by the algorithm in the respective curve (in μm). The margin of conformity between all curves is marked with a cross. The mean value is determined as the output value.

FIG. 8: Examples for measuring color intensity. Detail photos of a section scan with histogram. Immunohistochemistry for Melan A (red color substrate in the positive image), here is the inverse image, on which the brightness value in the corresponding green color channel is determined. Slice thickness A 7 μm, B approx. 3 μm slice thickness. The higher slice thickness shows a correspondingly higher color intensity.

FIG. 9: (A) Calibration cone with liquefied tissue (brain), which was diluted in a standardized manner, re-solidified in gel and embedded in paraffin. For each block, the concentration of the target protein (S100) was determined in a parallel approach in clinical chemistry. (B) Re-composition of these calibration cones in different concentrations C1 to C3 into a new common calibration block (diagram).

FIG. 10: Diagram of a slide 1 which can be used for measuring the section thickness and determining the concentration of a target substance in a specimen section 3. In the control region 2, a section of a calibration block (calibration specimen) is applied, which contains calibration cones (A-D), each of which contains a specific concentration of the target substance to be determined. The specimen section 3, which comprises a tissue sample 4, is applied to the central region of the slide. Calibration punches 7 can be introduced in the specimen section 3 and in the calibration specimen. Information for identifying the sample and the staining, for example, can be applied in the data field 6. The slide is inserted into the apparatus according to the invention. The section thickness is then measured in the regions marked in dark gray and/or in the regions of the calibration punches 7. In a preferred embodiment, a measurement is carried out at four measuring positions 5 in the region of the specimen section.

FIG. 11: (A) Calibration curve, created using the tonal values determined for the calibration blocks, which allows each tonal value to be clearly assigned to a concentration (the concentrations were determined in parallel in the same dilution series in clinical chemistry). x-axis: dilution series (10×10^{-x}) (B) Inverse image of the immunohistochemistry for S100 of the patient sample on the same section. A concentration of S100 can be assigned to each region of the image using the calibration curve and individual calibration. (C) shows the measured values that were determined for two adjacent tumor cells of a malignant melanoma (metastasis). At least one image pixel is required for the measurement, but ideally a plurality.

FIG. 12: Section of a calibration block (calibration specimen) containing three differently concentrated calibration cones of brain tissue, stained with immunohistochemistry for S 100 (brown color reaction).

FIG. 13: Correlation of the tonal value measured in a digital scan of the calibration specimen (inverse image, blue color channel) with the S100 protein concentration contained in the calibration cones.

FIG. 14: The mean values of the values measured at the four measuring positions (“North”, “East”, “South” and “West”) (scattering and absorption) show constantly decreasing measured values (y-axis, in % of the interpolated blank value) as a function of the wavelength (infrared, red, blue and violet) and the section thickness in μm (x-axis).

FIG. 15: Series of sections (1-7 μm) of a paraffin block containing a sphere of colored wax with a known color concentration (e.g. red), from which the calibration punches are also obtained. It can be seen with the naked eye that the color density increases with the thickness of the section.

FIG. 16: Dependence of the color intensity (brightness value, also referred to as tonal value) of the wax spheres described in FIG. 15 on the section thickness of the section series in μm (u) (1-7 μm, inverse image, green color channel).

FIG. 17: Unstained paraffin-embedded sections of varying thickness (top (calibration specimen) from left to right 7, 5, 3, 1 μm; bottom (specimen section) 1, 3, 5, 7 μm) with calibration punches contained and cut therein.

FIG. 18: Standard curves (mean values of different measurements) generated from the test section series (1, 3, 5 and 7 μm) respectively for diluted (right) and undiluted (left) wax.

FIG. 19: Staining of the test section series using immunohistochemistry for S 100. Tumor tissue at the top, calibration blocks at the bottom (adjusted section thickness of the microtome marked on the slide).

FIG. 20: Calibration curves: Left: calibration of the calibration specimen, right: for the specimen section (tumor sample) after correction of the difference in section thickness between the calibration specimen and the specimen section.

FIG. 21: Is an illustration of a preferred embodiment of the apparatus according to the present invention in an isometric view. The apparatus comprises shaping structure 8 (housing), into which a light source 9 (for example a light emitting diode) and a sensor 11 (for example a photodiode) can be introduced in opposite positions. The slide 1, onto which a specimen section and/or a calibration specimen is applied, can be placed between the light source 9 and the sensor 11 in such a way that the light beam emitted by the light source 9 is directed through the slide 1 and the scattering and/or absorption of the light beam can be measured by the sensor 11.

FIG. 22: Is a top view illustration of a preferred embodiment of the apparatus according to the present invention. The apparatus comprises one or more apertures 12 (position of the apertures indicated, apertures themselves not shown in the figure), which can be introduced into the shaping structure 8 to limit a light beam from the light source 9 (not shown in the present figure, shown in FIG. 23) to one or more specific positions of the slide 1 onto which a specimen section and/or a calibration specimen are applied, and thus to measure the scattering and/or absorption of the light beam by the specimen section or calibration specimen.

FIG. 23: Is a front view illustration of a preferred embodiment of the apparatus according to the present invention. The apparatus further comprises a camera 13 (position of the camera shown, camera itself not shown) which can record the position of the slide 1 illuminated by the light source 9 and is used to check the positioning of the one or more apertures 12 between the light source 9 and the slide 1 and between the sensor 11 and the slide 1 and thus enable measurement of the scattering and/or absorption of the light beam by the specimen section or calibration specimen applied to the slide 1 at one or more specific positions. Furthermore, the camera can be used to create an image of the specimen section or to determine further properties of the specimen section, for example to determine the homogeneity of the specimen in the form of measured values, which can be used to determine the section thickness or a concentration of a target substance in a specimen section. The light source 9 and/or the sensor 11 can be introduced on an insert 10 into the shaping structure 8 of the apparatus and can thus advantageously be exchanged by removing the insert and inserting an insert which comprises a different light source (for example a light-emitting diode of a different wavelength), thus enabling, for example, a measurement at a different wavelength.

FIG. 24: Is a side view illustration of a preferred embodiment of the apparatus according to the present invention. As described in FIG. 23, the light source 9 and/or the sensor 11 can be introduced on an insert 10 into the shaping structure 8 of the apparatus and can be interchanged. This makes it possible, for example, to measure the scattering and/or absorption of a light beam by the specimen section or calibration specimen, which are applied on the slide 1, at different wavelengths by exchanging the light source 9 for a light source 9 of a different wavelength.

FIG. 25: Images from the Lascerscan microscope. (A) Light image of the edge of a specimen section with height profile lines drawn in from the 3D scan, (B) the height profile determined along the arrow from the top right to the bottom left edge. A section thickness of 7.56 μm was determined.

FIG. 26: Summary of the measured values of all height profiles of the paraffin-embedded sections from the laser coherence tomography measurement, (A) individual values, (B) mean values. On the one hand, a strong scattering (A) of the measured section thicknesses is recognizable, on the other hand a large deviation of the mean values (B) upwards is recognizable compared to the nominal section thickness (setting of the microtome), especially for thin sections.

FIG. 27: Similarity of the scans. (A) After an image registration of the type “rigid” (translation & rotation) to compare the images, they could be superimposed using rigid body transformations (translation, rotation, no stretching). Subsequently, 10×8 measurement windows (squares) were defined. (B) The measured color values (from 0-250) are shown for each color channel and each window (1-80). It can be seen that the error bars/standard deviations in each window are very small.

FIG. 28: (A) Original scan with calibration punches (circles) and protein test substance (area, dark gray). (B) Determination of the mean color value (without consideration of the white surrounding area) with calibration punch top center.

FIG. 29: (A) Correction area spanned by the calibration punches (in color values) at the respective positions in the section. (B) False color image correction area.

FIG. 30: (A) Corrected image within the correction area (dashed line), (B) Highly intensified image to illustrate the unevenness and differences in section thickness within the section.

FIG. 31: Tissue preparation (brain tissue) with Histogel, cast, sectioned, paraffin embedded. (A) Immunohistochemistry for S 100: High degree of homogeneity of the material without recognizable systematic concentration gradients; small irregularities due to air bubbles and suspected sectioning artifacts. (B) Immunohistochemistry for NeuN, which labels neurons in a punctate manner, also here generally very homogeneous distribution.

FIG. 32: Example of a calibration cone (tissue preparation thickened in a defined dilution, HE staining). The calibration cone shows a high degree of homogeneity of the contained tissue.

FIG. 33: Tissue (unfixed brain tissue) after passing twice through an ultrafine sieve (400 mesh). Macroscopically (A) and also microscopically (B) (HE staining, specimen section after embedding), individual fragments can no longer be distinguished, the cell nuclei (dark dots) and fibers (everything else in between) appear almost homogeneously distributed.

FIG. 34: Measured values of the concentrations of S 100 (clinical chemistry) in dilution series of 3 different preparations that were made directly one after the other from the same sample material. The variance of the results is very small.

### EXAMPLES

### Paraffin-Embedded Section

Formalin-fixed and paraffin-embedded tissue is cut manually on the microtome. The section thickness is mechanically adjusted to a thickness of between 1 μm and 9 μm, usually 1 to 5 μm. During cutting, the section usually curls strongly. This effect is further enhanced by the tissue contained in the cut. After cutting, the section is stretched on a water surface and transferred from there to a slide.

The actual section thickness usually deviates greatly from the set section thickness and at the same time has an influence on the color intensity. This effect is based on the optical density of the specimen section increasing with the increased section thickness and the Beer-Lambert law. The influence of the section thickness on the color intensity can already be observed in unstained specimens based on the natural staining of the tissue (FIG. 1 natural pigments hemosiderin and melanin, FIG. 15Error! Reference source not found., histologically stained section).

### Accuracy of the Section Thickness Set on the Microtome

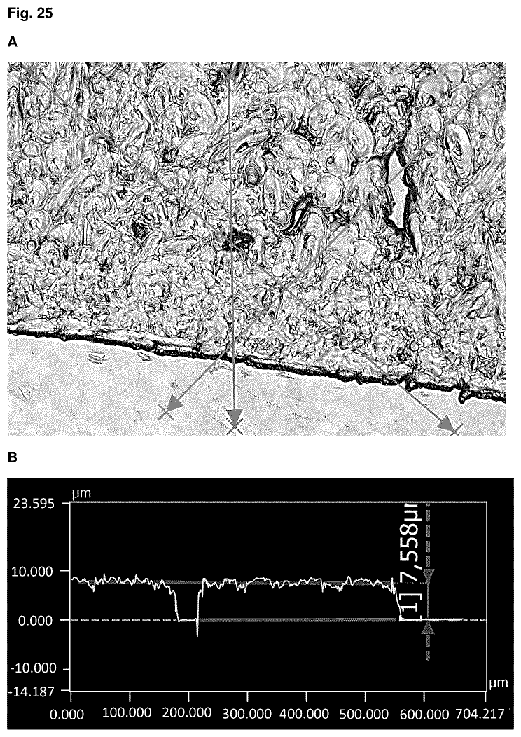

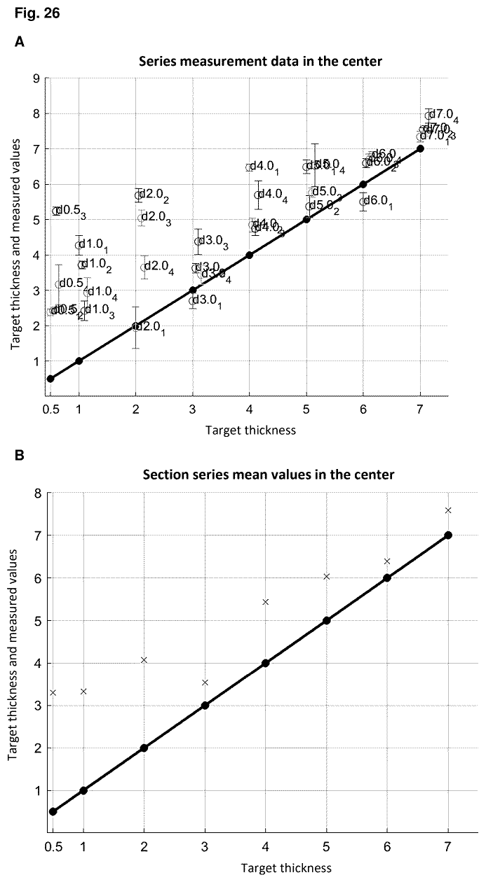

First, a standardized series of sections was produced on the test device (Leica RM2125 RTS microtome) from a test block consisting only of paraffin (without tissue): Four sections each with the section thicknesses set on the device 0.5 μm, 1 μm, 2 μm, 3 μm, 4 μm, 5 μm, 6 μm and 7 μm. An ultra-precise height profile and a 3D scan were made of each of these sections at the edge and in the center using a laser scanning microscope (Keyence VK-X1050 coherence tomograph). These were used to measure the height of the cut at the edge and in the center with an accuracy of approx. 0.01 μm. This showed that the height of this profile deviated considerably from the set thickness, in some cases by a multiple of the set thickness, particularly at low cutting thicknesses (see FIGS. 25 and 26). From this it can be concluded that there are complex folds in the paraffin-embedded section.

Determination of Section Thickness by Means of Scattering and/or Absorption

### Comparative Example—Transmission in the UV-C Range

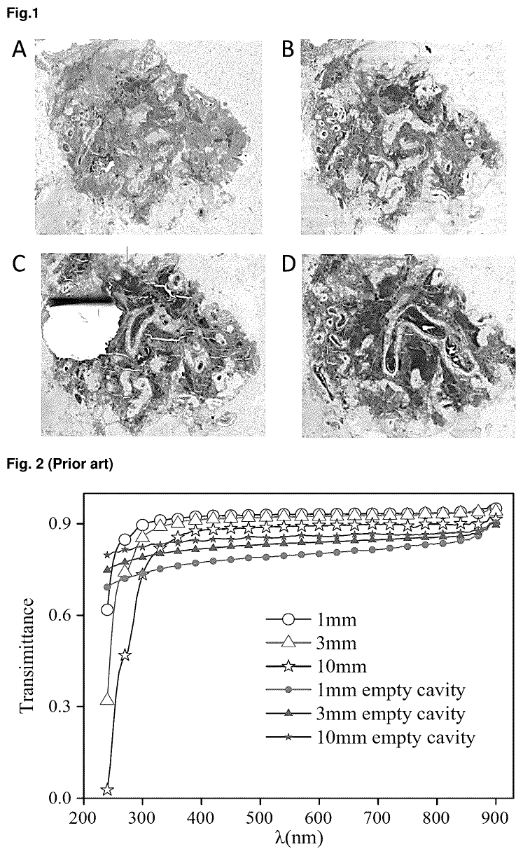

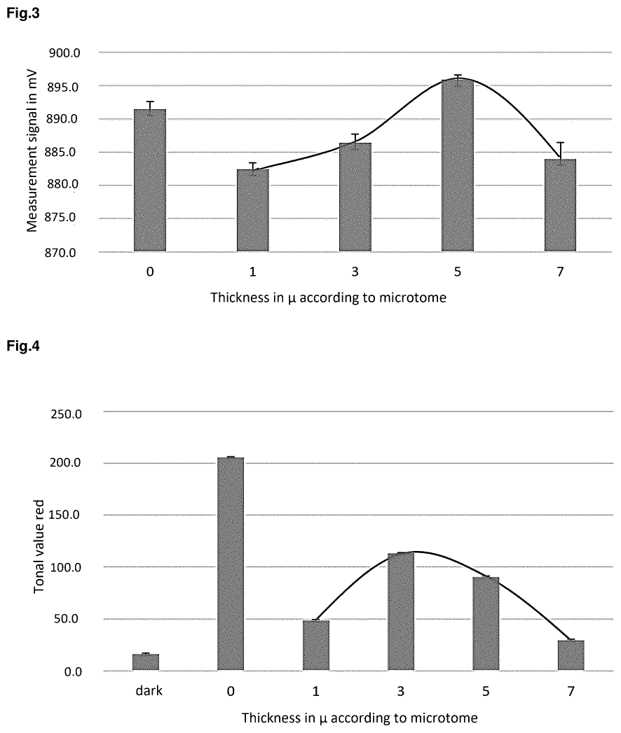

Transmission describes the relative transmittance of a medium (in this case paraffin) as a percentage and is reciprocal to absorption (absorption of radiation when passing through a medium). This varies depending on the wavelength of the electromagnetic radiation and is described in transmission curves. FIG. 2 shows the transmission curves for paraffin (according to Li et al. 2015).

It can be seen that paraffin has good light transmission from a wavelength of 300 nm, regardless of the section thickness. In the very short wavelength range (near 200 nm and below), paraffin becomes opaque for all measured section thicknesses. In the limit range between 200-300 nm, however, there is a strong sensitivity of the measurement (i.e. variability of the measurement result with changing input variable) depending on the section thickness.

A circuit with a UV-C sensitive photodiode was selected for a measurement in this wavelength range. A commercially available UV-C fluorescent tube (OSRAM Puritec HNS 5 W), whose emission spectrum shows a strong peak at 254 nm, was initially used as the light source. Slides with paraffin-embedded sections with section thicknesses of 1, 3, 5 and 7 μm set on the microtome were placed in the beam path between the radiation source and the sensor, initially without a tissue sample. A section of the slide without paraffin was measured as a blank.

For measurements in the UV-C range, it was necessary to use high-purity quartz glass (synthetic fused silica). Standard slides can be used for all other wavelengths.

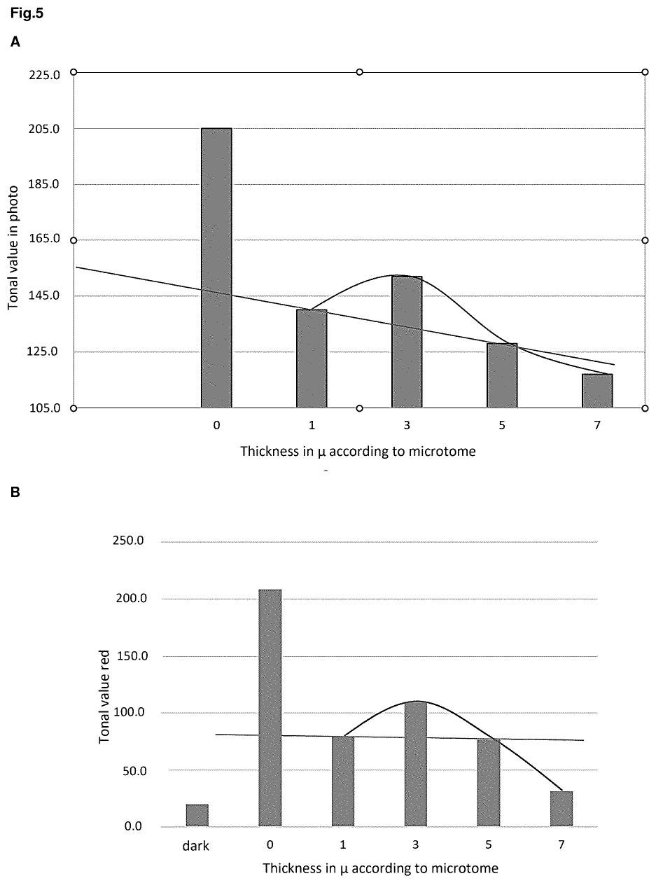

The results of the measurements are shown in FIG. 3 as mean values of the extinction with UV-C as the radiation source in mV of the sensor (sensor type: UVC-T10GM-LA, UV sensor module, 220-280 nm).

Contrary to expectations, there was not a continuous decrease in the measurement signal with increasing section thickness, but rather a curve. The maximum of the curve lies at an average coating thickness of 5 μm and exceeds the blank value. This may be due to a possible fluorescence effect, which often occurs with UV radiation. In the case of this fluorescence effect, a radiation emission is caused by an originally shorter wavelength of a radiation source. As a result, the emission of the sample appears stronger than would be expected based on the intensity of the actual radiation source (known effects with signal colors, whiteners or so-called black light) and can therefore exceed a blank value. Furthermore, scattering effects and reflections in the measuring chamber can be of significance. As a substantial hypothesis from these considerations, it was assumed that a second effect plays a role in addition to absorption at low section thicknesses. Scattering was investigated as the most likely second effect after absorption.

### Example According to the Invention—Scattering and Absorption

Scattering describes the splitting of a light beam (deflection of photons) due to irregularities in a medium that is in principle translucent (e.g. fog). A paraffin-embedded section appears cloudy to the naked eye and only dimly translucent. The clouding of a paraffin-embedded section can be explained, for example, by the formation of unevenly distributed solidification regions when the liquid paraffin is cooled and the resulting transitions at which scattering occurs. Scattering in an optical medium leads to a reduction in image contrast. Bright points of light are therefore attenuated to an above-average degree. Initially, a laser light source seemed particularly suitable for the measurement. This was not aimed directly at a sensor, but at a projection surface (matt photo cardboard). This had the advantage that surrounding scattering patterns were also recorded. The projection was captured in a standardized way with a digital camera (same exposure data for each photo, no automatic exposure or sensitivity). The brightness or attenuation compared to the blank (slide without paraffin-embedded section) was recorded as the tonal value of the photo in the corresponding color channel (red, 0-255). Based on this, the apparatus described below was developed, in which a direct measurement with photodiodes is carried out.

The method demonstrated very good repeatability with multiple measurements in different segments of paraffin-embedded sections and highly significant differences between different section thicknesses. It was assumed that the scattering increases uniformly with the section thickness. However, in the same way as with the UV-C measurement, a curve was produced with a maximum at an average section thickness of 3 μm (FIG. 4). The conclusion from this was that the scattering is particularly high at low section thicknesses and therefore the transmittance is reduced more strongly at low section thicknesses and causes the curve shape with a maximum. Microtography and laser microscopy contributed to the clarification: These actually show a stronger folding of the section at low section thicknesses (not shown). All effects together are taken into account in the modeling.

Another observation was that the maximum of the curve occurs at different section thicknesses depending on the wavelength of the light source. While the maximum for the measurement in the UV-C range was at a section thickness of 5 μm, the maximum for red laser light (650 nm) was at a section thickness of 3 μm.

In the following, further measurements were carried out with radiation sources of different wavelengths. Lasers with blue (λ approx. 450 nm) and violet light (λ=405 nm) and an infrared source (A approx. 980 nm) were used.

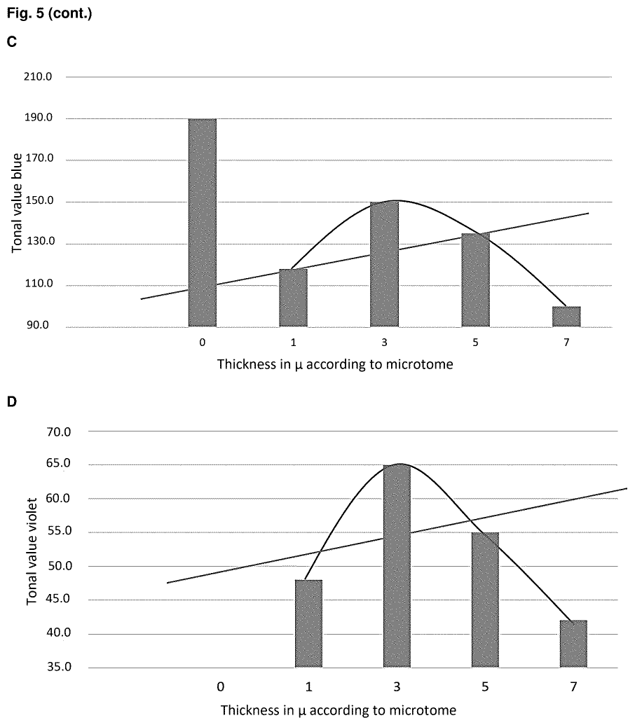

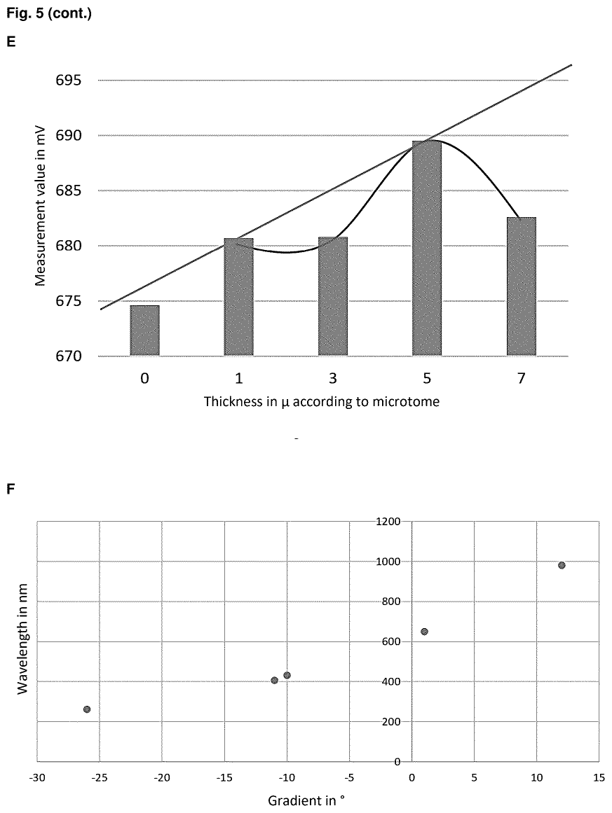

Differences in the maxima of the curves exist in particular between section thicknesses of 1 μm and 5 μm (FIG. 5). The 1 μm section produces significantly higher values in the infrared range than the 5 μm section, and the reverse is true for the violet light source (405 nm). To visualize the effect, a straight line was drawn between the measured values of the 1 μm and 5 μm section thicknesses, the angle of inclination was recorded and plotted against the wavelength of the radiation source. A clear and regular effect can be seen here, which is also visible with small differences—for example between blue and violet light. As the maxima of the curves are far enough apart, both measurement at one wavelength and the combination of measurements at different wavelengths can provide a clear retrograde assignment to the section thickness with sufficient accuracy. Measurement at a plurality of wavelengths increases the accuracy.

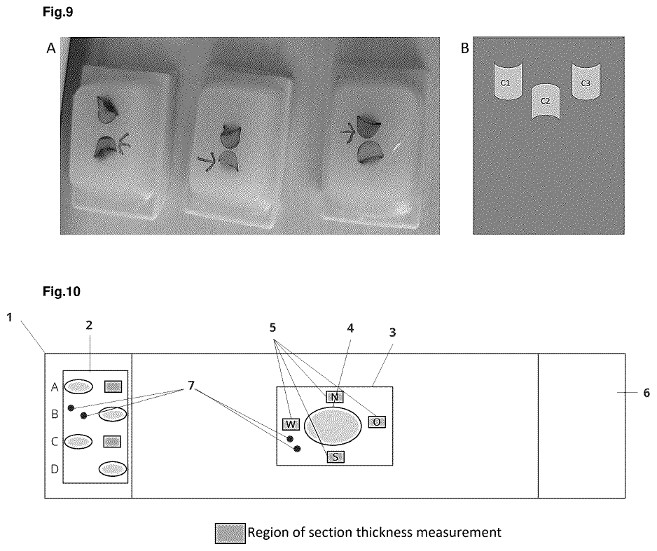

Paraffin-embedded sections with contained tissue often exhibit irregular structures. These irregularities can be made visible in scans of unstained specimen sections or under a laser microscope. Accuracy can be improved by “multi-position measurement” of a section at different positions on the section. For this purpose, scattering and absorption were measured at different wavelengths (infrared to violet) at four defined positions (“North”, “East”, “South” and “West”, see FIG. 10) on the paraffin-embedded section, which contains unstained tumor tissue. As previously observed, the transmittance decreased with increasing section thickness. The mean values of the four measurement positions around the tissue demonstrated a clear dependence on the wavelength and the section thickness (FIG. 14). It should be noted that the curve of paraffin-embedded sections containing tissue shows a different course than those not containing tissue (no maximum), which must be taken into account during calibration.

### Example According to the Invention—Absorption

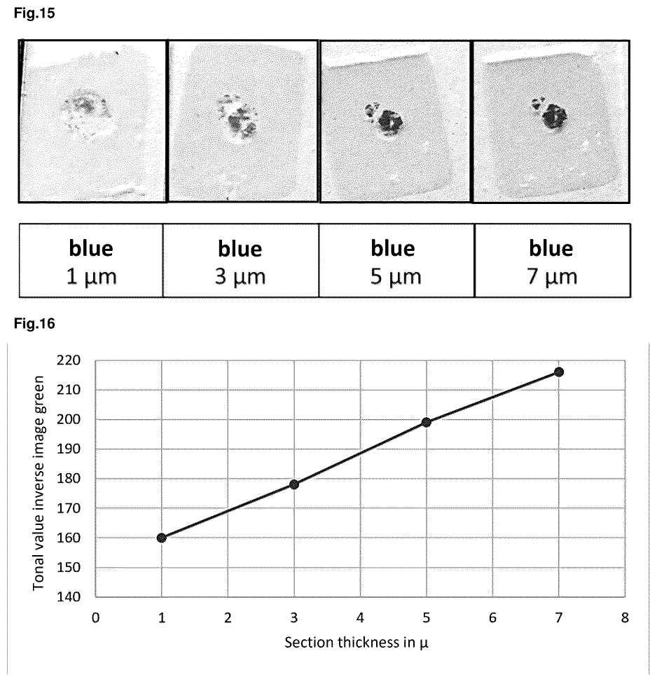

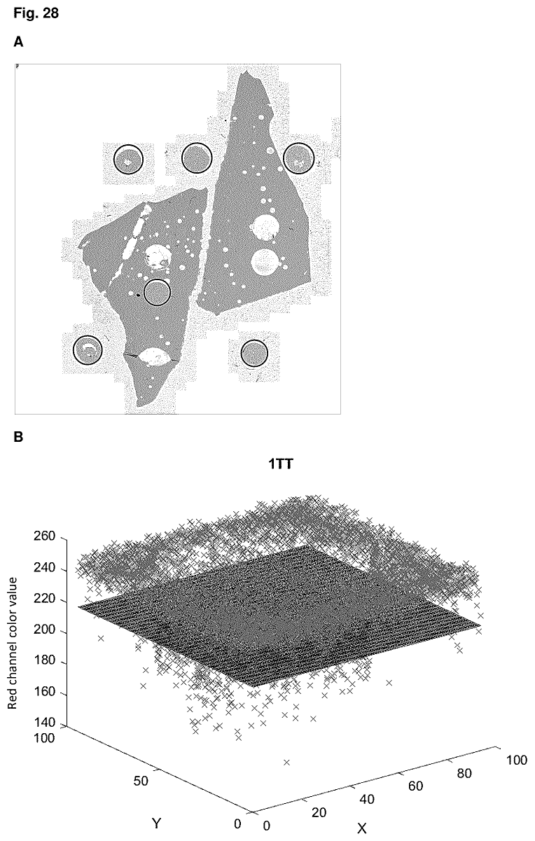

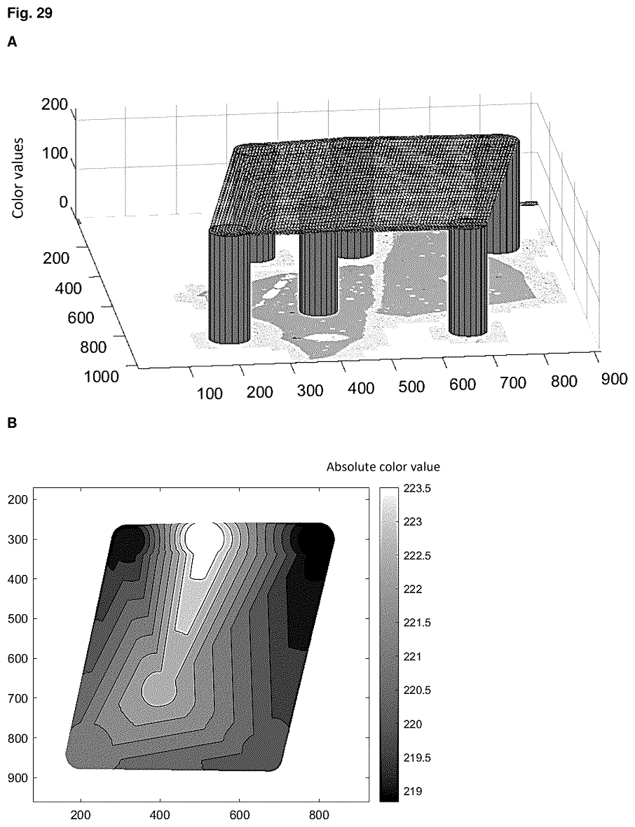

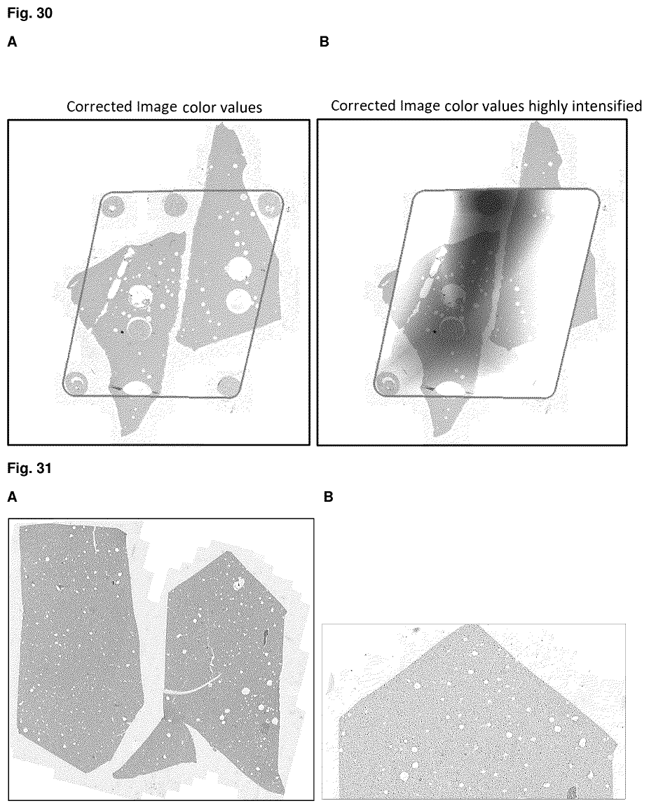

The accuracy can also be increased by inserting so-called “wax punches”, also known as “calibration punches” (regions containing a dye that absorbs in the wavelength range from 200 to 2000 nm). First, balls of stained wax (in this case red) with a known color concentration were poured into a new paraffin block. A series of sections (1-7 μm) was produced from such a block. It can be seen that the color density and thus absorption increases with the thickness of the section (FIG. 15). The sections were scanned and the color density was determined as a tonal value (0-255). This demonstrates a clear linear dependence of the values on the section thickness (FIG. 16). By inserting one or more calibration punches into a specimen section, the section thickness can be determined by measuring the absorption in these areas. Using a plurality of calibration punches made of stained wax in different regions of the specimen section, a height profile of the section can also be created. With this information, a correction factor can be determined for each region of the section and the scan of the staining can subsequently be corrected accordingly for each region (FIGS. 28 to 30).

Script for Modeling the Section Thickness from Calibration Values

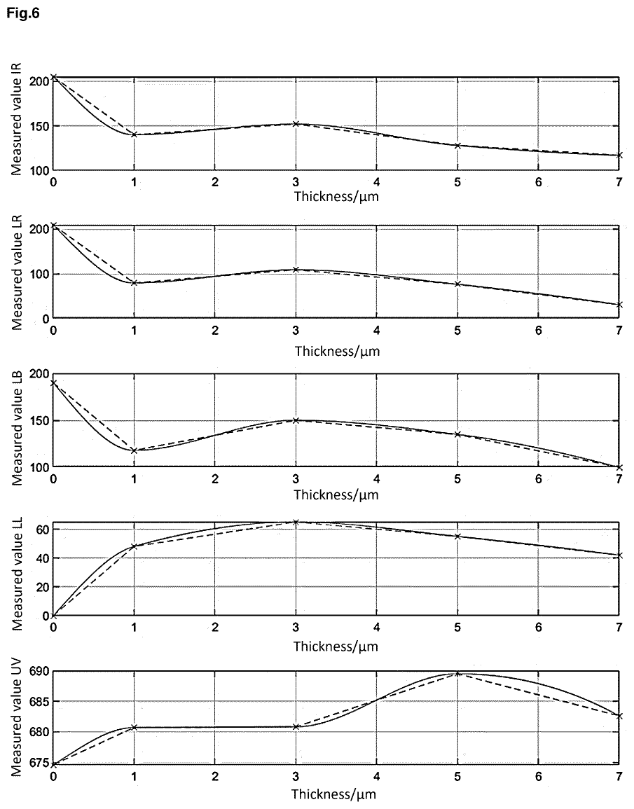

An evaluation script was developed in the Matlab software to evaluate the section thickness from the obtained measurement values (calibration values) (scattering and/or absorption). This performs a fitting of each individual curve for different light wavelengths with the designations IR (980 nm), LR (650 nm), LB (450 nm), LL (405 nm) and UV (260 nm) with a smoothing spline function (piecewise interpolation function that includes each interpolation point). The curves are shown in FIG. 6. This script initially applies to paraffin-embedded sections not containing any tissue.

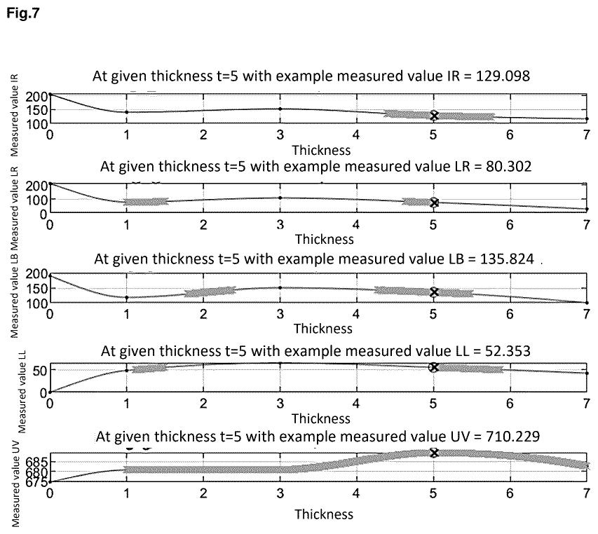

The software uses the scattering values measured for a paraffin-embedded section in one or more of the 5 curves to determine the actual section thickness. For this purpose, the respective scattering value (separately for IR, LR, LB, LL and UV) is applied with the known measurement uncertainty (e.g. ±5%) as the measured value range. The values of the curves that lie within the associated range are then marked in the curve (FIG. 7). These are the possible section thicknesses for which the measured value can be measured within the measurement accuracy and which are calculated by the script as being plausible section thicknesses for the paraffin-embedded section. The accuracy of the determination can be increased by using a plurality of curves measured at different wavelengths.

If a plurality of curves are used, the matching thicknesses of the sections (marked with a cross in FIG. 7) result in a range of possible section thicknesses. The section thickness of the paraffin-embedded section is determined by calculating the mean value and the standard deviation of these possible section thicknesses.

In the following, “artificial measured values” were generated for various thicknesses, for which the quality of the evaluation was compared. A test section thickness (“real section thickness”) was defined for this purpose. This corresponds to the measurement of the section thickness of a section that is to be analyzed using the script. Based on this, “model measured values” were generated, i.e. measured values or sensor outputs to be expected for the given section thickness according to the known curves. To account for a random error in the measurement, a random “measurement error” of 5% was applied to these values (pseudo-random number with the Mersenne twister generator and a seed of 1).

The evaluation over a section thickness range of 1-7 μm results in a high accuracy of the script over a wide range. Deviations occur in the range of 2-3.5 μm. If the section thickness set on the microtome (“target section thickness”) is known, the deviations in this range are significantly reduced.

### Measurement of Color Intensity

The first step was to create a digital scan of the sections.

The scanner generates an image that is equally bright in all segments with the same white balance and is always carried out in the same way thanks to the calibration, even with different specimen sections. To measure the color intensity, the tonal value of the closest color channel (red, green or blue) is determined in an image processing program (if necessary using the inverse image).

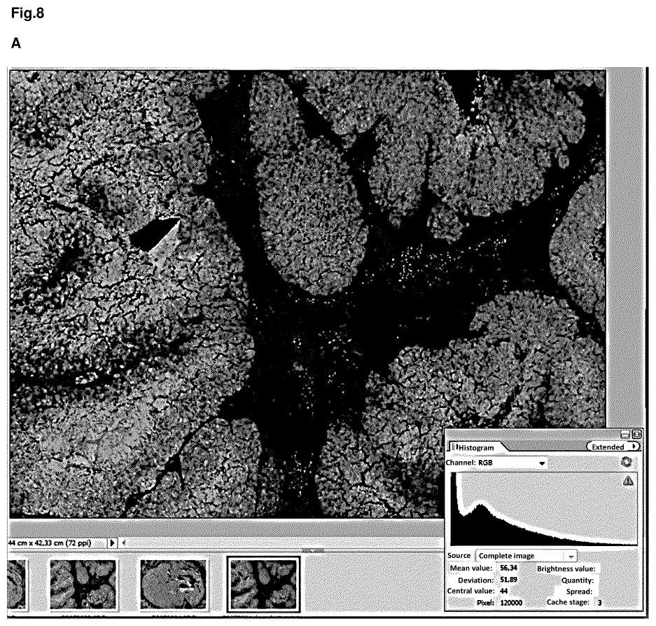

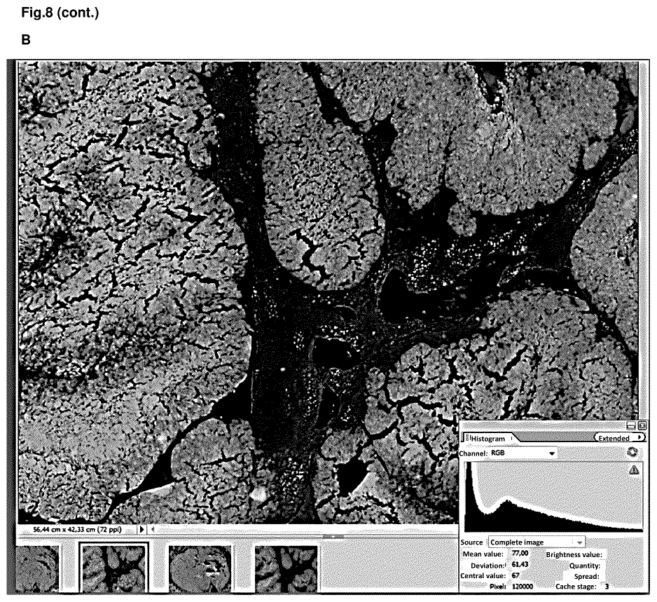

Further series of sections with 1, 3, 5 and 7 μm with tissue were prepared and the density of the color of the tissue was determined as the tonal value/intensity of the color (0-255) on the digital scan. Initially, natural pigment was used for orientation. A clearly monotonic relationship was recognizable, i.e. color intensity steadily increasing with the section thickness. In further test series, this was then also carried out using immunohistochemical staining (FIG. 8).

The principle can be applied to any substance if it has an inherent color or can be stained. As natural colors (e.g. the blood pigment hemoglobin) are usually too pale for the very low section thicknesses used, histochemical staining is generally used. In addition, an optical kit (mounting medium) is inserted between the tissue and the cover glass. This minimizes scattering by filling the irregular surface and enables a uniform image that can be assessed without interference.

### Accuracy of the Specimen Scan

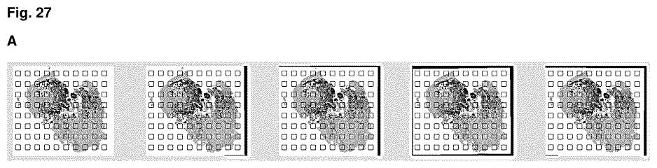

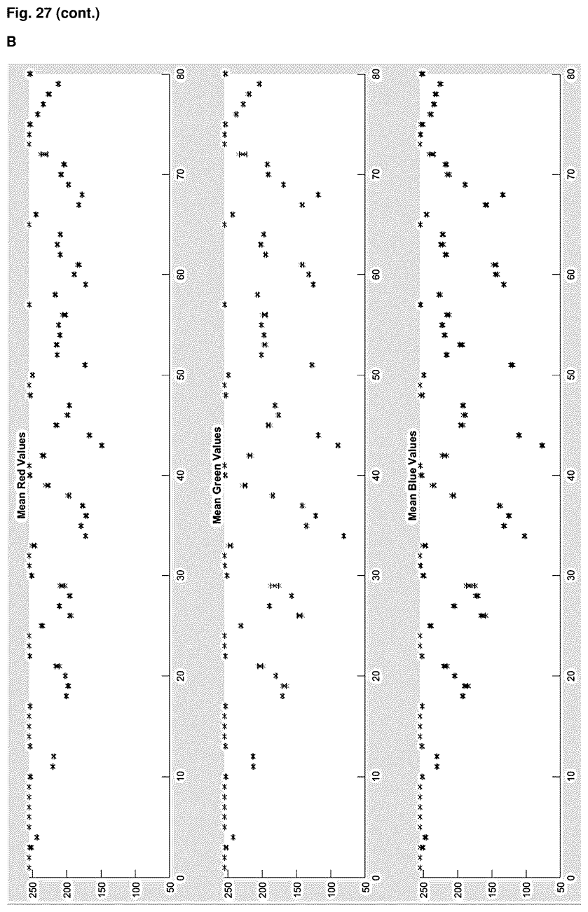

For this purpose, a stained test specimen with different structures and partly natural tissue pigments was scanned 5 times on the same device (Pannoramic Desk). The color density of the scans was measured at defined areas. The accuracy was high (the scattering range was low) (FIG. 27).

### Calibration of the Concentration Determination

To establish the method, a protein was used that can be examined in histological sections and with the methods of clinical chemistry, in both methods as standard, and with high accuracy and can be reliably extracted from tissue.

The protein S100 is a calcium-binding protein with a low molecular mass (9-13 kDa) with different subtypes; the subtype S100B was detected here. It is mainly expressed in glial cells, but also in other tissue types such as melanocytes and melanocytic tumors. A fresh (unfixed) human cerebrum sample was used as tissue.

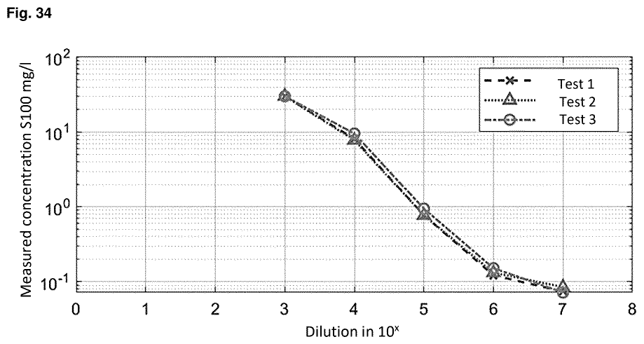

For clinical chemistry, the tissue was mechanically homogenized by blending, largely dissolved with a lysis buffer and then strongly diluted (105-fold) in order to reach the usable measuring range, which is very low with this method (between 0.02 μg/L-30 μg/L) (FIG. 34).

In histopathology, the process was slightly different: Since a histological preparation undergoes a plurality of rinsing steps during preparation, buffers without lysing substances were used here so that the tiniest tissue fragments were available in the same dilution as in clinical chemistry for the calibration specimens. The tissue was mechanically ground with a blender in a small amount of liquid and mechanically bound in a gel with a defined dilution (“calibration cone”). Alternatively or additionally, the fresh tissue can be passed through a very fine sieve (100 mesh, i.e. 100 meshes per inch) and mixed with histogel to solidify. To achieve even greater homogeneity, the tissue can also be passed once or a plurality of times through a 400 mesh sieve (FIG. 33).

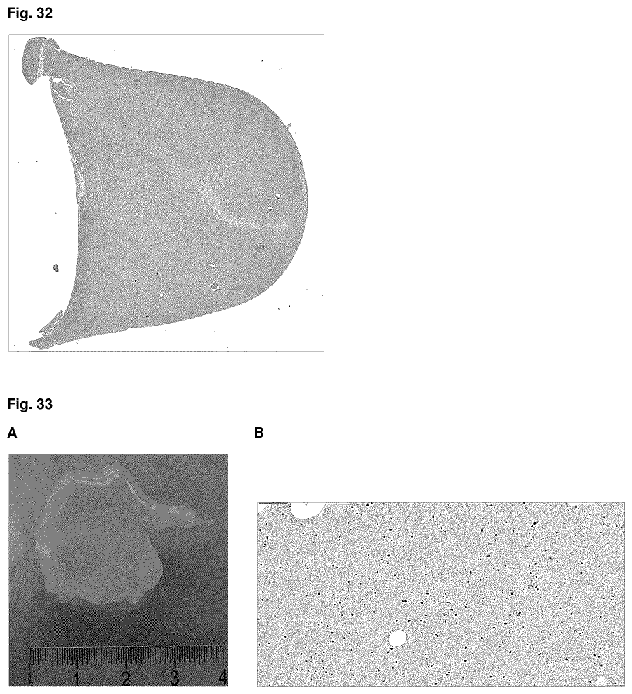

This calibration cone was embedded in paraffin and cut, wherein a homogeneous distribution of the tissue within the cone was revealed (FIG. 32). A plurality of cell blocks with a defined dilution were produced. The tissue concentration contained in these cell blocks was calculated by comparison with clinical chemistry. These cell blocks were then poured into a common new paraffin block as a calibration instrument (“calibration block”) (FIG. 9). Furthermore, recombinant test protein provided by the manufacturer can also be injected directly into the calibration cone in a high concentration as a small deposit of approx. 1 mm in size).

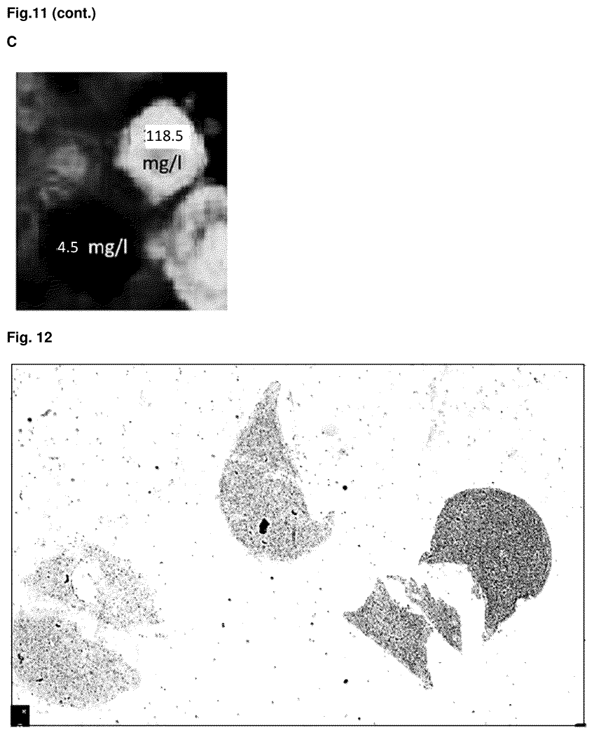

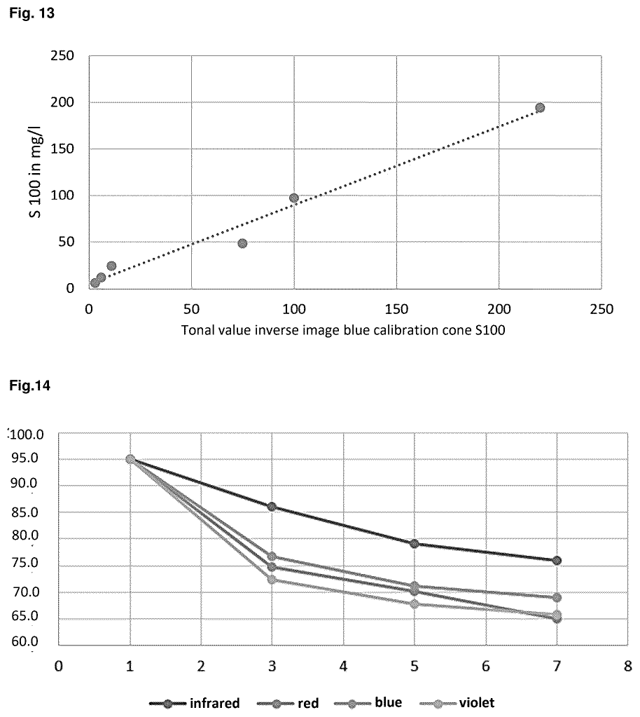

A specimen section was prepared from the cell block and immunohistochemistry was performed for S 100. This was scanned and the color concentration was determined as the tonal value of the image between 0-255 (blue color channel in the inverse image or brown coloration in the positive image). Each tonal value can be clearly assigned to a concentration of S100, allowing a calibration curve for S100 to be created using the calibration specimen (FIG. 12, FIG. 13).

### Staining the Specimen Using Immunohistochemistry

A possible source of error in determining the concentration of a target substance in a tissue is the distribution of the antibodies on the tissue section. To test this, prepared tissue was poured into an approx. 5×6 cm mold as described above to ensure a high degree of homogeneity during solidification. After formalin fixation, this casting plate was cut into orientable strips, paraffinized and poured into paraffin blocks. These were routinely stained for the target protein (S100) in an area staining procedure, as well as in a spot staining procedure (in this case NeuN for the neurons evenly distributed in the tissue preparation). The staining showed only minimal variation (FIG. 31).

### Application of the Complete Method—Scattering and Absorption

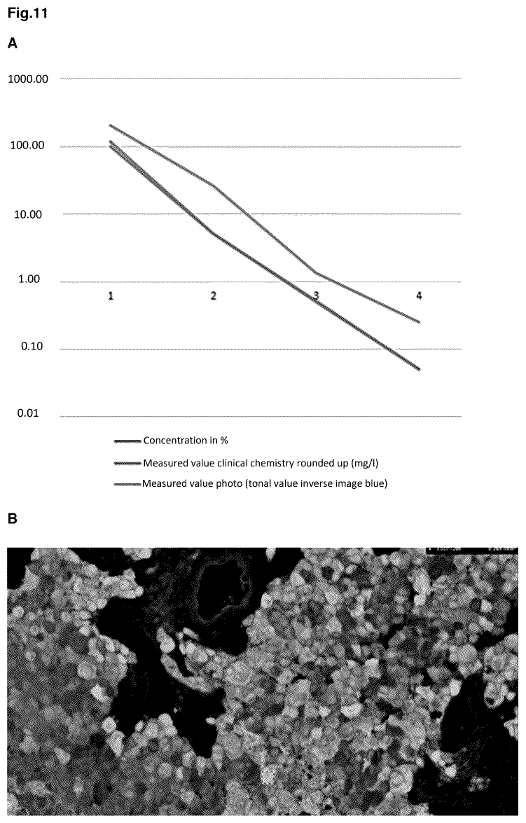

For the final test of the method, a tissue sample was used that was known to express S 100: The metastasis of a malignant melanoma. The slides were prepared by applying a section of the calibration block with anonymized section thickness to one half of the slide. A section of the tumor sample was applied to the other half, also with anonymized section thickness (FIG. 10). For both sections, the section thickness was determined in the tissue-free edge of the section using the method described above and the correction factor was determined. The section was scanned and representative photos were taken. The color intensity (tonal value, inverse image blue) was determined for each concentration on the calibration sections and an individual calibration curve was calculated from these three values. The color intensity was also determined for the desired segments in the tumor and the concentrations of the protein S100 were directly assigned using the individual calibration curve and labeled in the photo (FIG. 11 and Table 1).

TABLE 1
Measured values of the calibration block determined in the method

| | | Measured value | Measured value |
|---|---|---|---|
| | | clinical chemistry | photo (tonal value |
| Dilution | Concentration in % | (mg/L) | inverse image) |
| 1 | 100 | 118 | 200 |
| 20 | 5 | 5.09 | 26.0 |
| 200 | 0.5 | 0.509 | 1.3 |
| 2000 | 0.05 | 0.0509 | 0.3 |

### Application of the Complete Method—Absorption

In order to precisely insert a wax punch (“calibration punch”) into the block to be measured, which contains the tissue to be examined, a method analogous to the creation of a tissue microarray was used. Using a hollow needle, tissue cylinders were removed from a paraffin block containing the stained wax and then punched into the block containing the tissue to be examined or the calibration cones. Two wax punches of different concentrations were punched into each block.

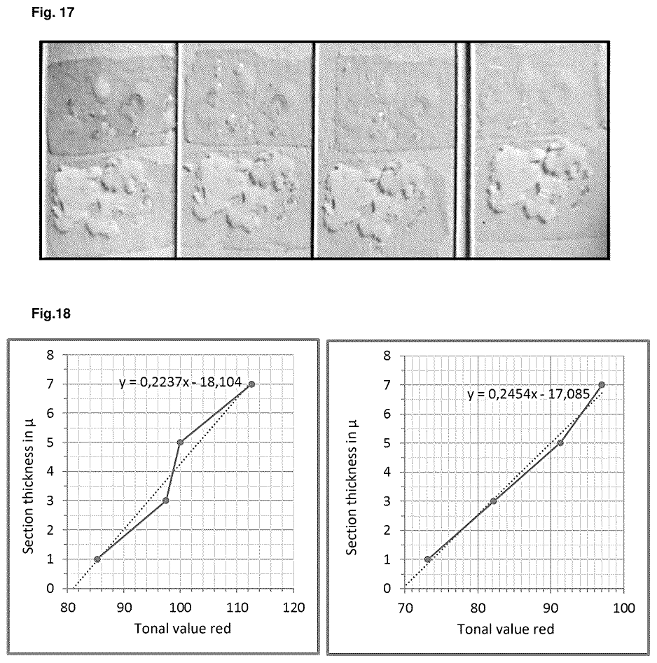

From these blocks, unstained series sections of different section thicknesses (1, 3, 5 and 7 μm) were first prepared and placed on a slide (FIG. 17). The unstained sections were scanned and the color intensity (brightness values) in the regions of the calibration punches was measured using the scan image. In parallel, the scattering of red laser light was photographed (in the region next to the tissue).

By determining the color intensity of the wax punches in the sections of different section thicknesses 1, 3, 5, 7 μm, a standard curve could be created for each of the two concentrations of the wax punch (FIG. 18). Based on this, a correction factor was determined between the section thickness of the calibration block and the block containing the tumor tissue.

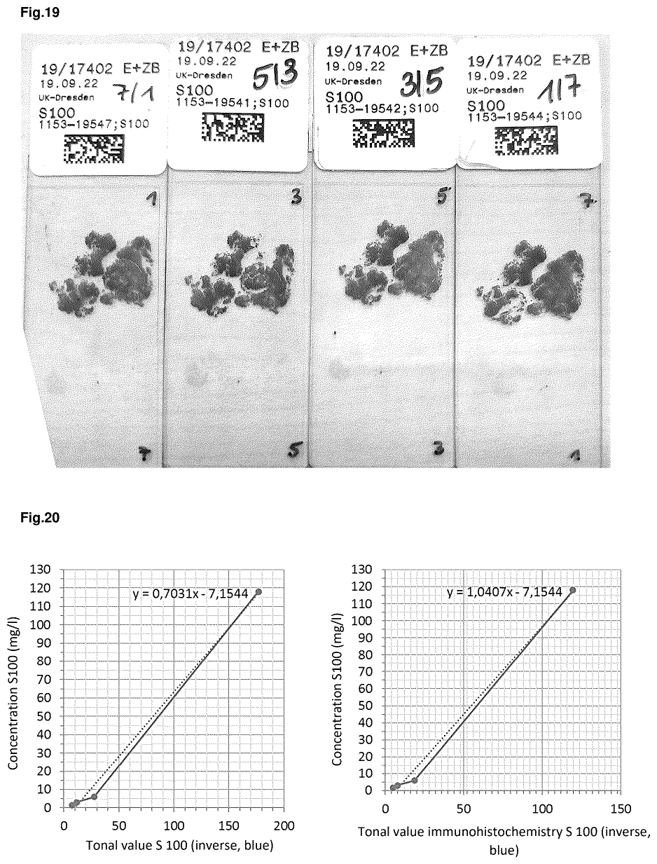

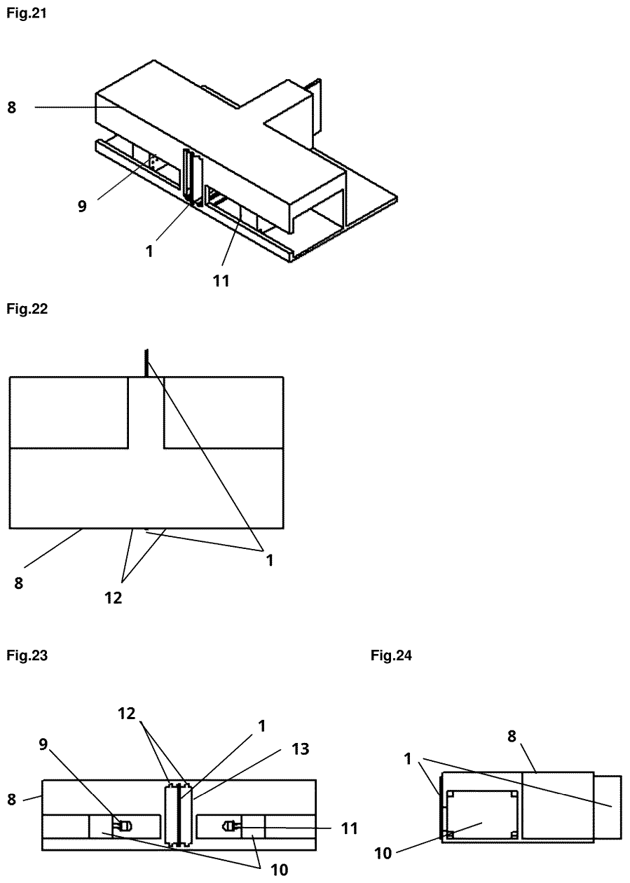

The sections were then immunohistochemically stained (FIG. 19). The calibration punches (in this case wax punches) dissolved and were no longer measurable. Alternatively, a calibration punch can be used, which contains a material that does not dissolve during staining and fixation of the sections. The stained sections were then scanned and the color intensity of the tumor tissue and the calibration cone of the calibration block were measured using the scan image. The measurement ranges are shown schematically in FIG. 10.

Based on the color intensity of the calibration cones in the calibration block and the correlation with the known concentration of the substance contained in the calibration cones (in this case S 100 protein), a calibration curve was created, which was used to determine the concentration in the tumor tissue of the specimen section based on the brightness value of the tumor tissue in the scanned image, taking into account the correction factor determined (FIG. 20). This can be displayed linearly in the core area of the application. For this purpose, a spreadsheet was created that summarizes all of the above calculations. The measured values (calibration values) must be entered into the spreadsheet or a corresponding device, from which the individual calibration is calculated. The measured values of the specimen section (the area of the tissue sample to be measured) are then entered and the concentration of the target protein is calculated and output.

1. A method for determining a section thickness of a specimen section, comprising
a. measuring one or more values generated from scattering and/or absorption of a light beam by the specimen section, using one light source, or multiple light sources of different wavelengths,
b. providing a modeling of the section thickness of the specimen section based on calibration values, wherein said calibration values are generated by measuring scattering and/or absorption of a light beam by one or more calibration specimens using one light source, or multiple light sources of different wavelengths, and
c. determining the section thickness of the specimen section by using the measured values according to (a) and the modeling according to (b).

2. The method according to claim 1, wherein the light source(s) exhibit a wavelength of 200 to 2000 nm.

3. The method according to claim 1, wherein the modeling of the section thickness comprises,
a. providing calibration values determined by measuring scattering and/or absorption of a light beam by one or more calibration specimens using one light source, or multiple light sources of different wavelengths, wherein the calibration specimens exhibit a known section thickness of 0.1 to 10 μm,
b. determining one or more calibration functions, wherein the calibration function comprises correlating section thickness of the one or more calibration specimens with the calibration values,
c. comparing the measured values of a specimen section with the one or more calibration functions according to (b),
d. outputting the section thickness of the specimen section corresponding to a value, or the mean value of multiple values, that match one or more of the calibration functions according to (b).

4. The method according to claim 1, wherein the specimen section exhibits a section thickness of 0.1 to 10 μm.

5. The method according to claim 1, wherein the specimen section comprises tissue.

6. The method according to claim 5, additionally comprising determining a concentration of a target substance in the tissue comprised in a specimen section.

7. The method according to claim 6, wherein determining the concentration of the target substance comprises:
a. providing a specimen section and a calibration specimen, wherein the calibration specimen comprises a predetermined concentration of the target substance,
b. determining the section thickness of the specimen section and the calibration specimen,
c. determining a correction factor by using the section thickness according to (b),
d. simultaneous staining of the specimen section and the calibration specimen,
e. measuring a color intensity of the tissue contained in the specimen section, and of the target substance contained in the calibration specimen, and
f. determining the concentration of the target substance in the tissue by using the color intensity according to (e) and the correction factor according to (c).

8. The method according to claim 7, wherein the calibration specimen contains the target substance in two or more concentrations which are spatially separated from each other.

9. The method according to claim 7, wherein the section thickness of the specimen section and of the calibration specimen is determined in tissue-free areas of the specimen section and of the calibration specimen.

10. The method according to claim 7, wherein the tissue-free regions of the specimen section and/or of the one or more calibration specimens comprise one or more regions which contain a dye which absorbs light in a wavelength range from 200 to 2000 nm.

11. The method according to claim 7, wherein the specimen section and the one or more calibration specimens are a paraffin-embedded section.

12. The method according to claim 7, wherein a digital scan is made of the specimen section and/or the calibration specimen, which is used for measuring values and/or color intensity, wherein measuring the values and/or the color intensity comprises determining a brightness value.

13. The method according to claim 7, wherein the measured color intensity of the target substance present in the calibration specimen is used to generate a calibration curve for the concentration of the target substance.

14. The method according to claim 13, wherein the target substance is a substance within the tissue comprised in a specimen section, and the target substance is localized in the tissue contained in the specimen section using the calibration specimen and the calibration curve, and the concentration of the target substance is determined.

15. An apparatus for measuring the section thickness of a specimen section and optionally for determining the concentration of a target substance in the specimen section, comprising
a. one or more light sources of different wavelengths in the range from 200 to 2000 nm,
b. at least one sensor for measuring scattering and/or absorption of a light beam,
c. an apparatus for positioning the specimen section during measurement, and
d. a computing unit for modeling the section thickness of a specimen section or for using the modeling in determining the section thickness of a specimen section and optionally for calculating the concentration of a target substance contained in the specimen section.

16. A slide comprising a calibration specimen containing a target substance in two or more concentrations which are spatially separated from each other which is suitable for a specimen section to be applied.

17. The method according to claim 2, wherein the light source(s) exhibit a wavelength of 300 to 1800 nm.

18. the method according to claim 4, wherein the specimen section exhibits a section thickness of 1 to 7 μm.

19. The method according to claim 10, wherein the tissue-free regions of the specimen section and/or of the one or more calibration specimens comprise one or more regions which contain a dye which absorbs light in a wavelength range from 300 to 800 nm.

20. The method according to claim 13, wherein the measured color intensity of the target substance present in the calibration specimen is used to generate a calibration curve for the concentration of the target substance, which is determined from two or more concentrations of the target substance.
